(19) United States
(12) Patent Application Publication
(10) Pub. No.: US 2022/0145285 A1
(43) Pub. Date: May 12, 2022

(54) Compartment-Free Single Cell Genetic Analysis

(71) Applicant: Cellecta, Inc., Mountain View, CA (US)

(72) Inventors: Alex CHENCHIK, Mountain View, CA (US); Russell Paul DARST, IV, Mountain View, CA (US); Lester KOBZIK, Mountain View, CA (US); Mikhail MAKHANOV, Mountain View, CA (US); Donato TEDESCO, Mountain View, CA (US)

(21) Appl. No.: 17/438,571

(22) PCT Filed: Aug. 12, 2020
(86) PCT No.: PCT/US2020/045865

§ 371 Date: Sep. 13, 2021

Related U.S. Application Data
(60) Provisional application No. 62/895,719, filed on Sep. 4, 2019.

(51) Int. Cl. C12N 15/10; C12Q 1/6876; C12Q 1/6804

(52) U.S. Cl. CPC C12N 15/1065; C12Q 1/6804; C12Q 1/6876; C12N 15/1096

(57) ABSTRACT

Compartment-free single cell genetic analysis methods are provided. Aspects of the methods include: (a) combining a cellular sample with a plurality of distinct barcoded beads comprising barcoded reverse primers under conditions sufficient to produce a liquid composition comprising a plurality of separated cell/barcoded bead complexes; (b) hybridizing template binding domains of barcoded reverse primers to template nucleic acids of the cells to produce primed template nucleic acids; and (c) subjecting the primed template nucleic acids to primer extension reaction conditions sufficient to produce barcoded nucleic acids, e.g., for subsequent amplification and analysis, such as by Next Generation Sequencing (NGS) protocols. Also provided are compositions that find use in practicing embodiments of the methods.

### CROSS-REFERENCE TO RELATED APPLICATION

Pursuant to 35 U.S.C. § 119(e), this application claims priority to U.S. Provisional Application Ser. No. 62/895,719 filed Sep. 4, 2019, the disclosure of which application is herein incorporated by reference.

### INTRODUCTION

Biomedicine has entered an era of advances at the cellular and molecular level. The goal of researchers and clinicians alike is to understand and then modify cell behavior through molecular techniques and tools. The methodologies for assessing cell biology at a molecular level are numerous. They include analyses of genomic DNA sequences, epigenetics, chromatin structure, messenger RNA (mRNA), non-protein-coding RNA, protein expression or modifications, and metabolites.

One area that has proven especially productive is the study of mRNA molecules (collectively termed the ‘transcriptome’), whose expression correlates well with important cellular features and with changes in cellular state. Transcriptomics was first applied to large pools of millions of cells, starting with hybridization-based microarrays, and later with next-generation sequencing (NGS) methods referred to as RNA-seq. RNA-seq on pooled cells has provided a vast amount of data that continues to spark discovery and innovation in biomedicine.

One limitation of the pooled approach is that the results represent an average of a large number of cells, often comprised of mixtures of cells differing from each other with respect to identity, phenotype and/or function. The nature of pooled cell studies does not allow detailed evaluation of the fundamental biological unit—the individual cell. This limitation has been addressed with the development of single cell genetic analysis methods, such as single cell DNA sequencing (scDNA-seq) or single cell RNA sequencing (scRNA-seq) approaches. scRNA-seq can identify and quantify RNA molecules in individual cells with high resolution and on a genome-wide scale. Indeed, use of scRNA-seq methods has increased rapidly. This reflects the recognition that biomedical researchers and clinicians can make important new discoveries using this powerful approach.

One major use of scRNA-seq has been to delineate transcriptional similarities and differences within a population of cells. For example, early studies revealed previously unappreciated levels of heterogeneity in embryonic and immune cells. Thus, the remarkable heterogeneity of seemingly identical cell populations remains a core reason for investigations using scRNA-seq.

Similarly, scRNA-seq can identify transcriptional differences between individual cells which allows identification of rare cell populations that would otherwise go undetected in analyses of pooled cells, such as malignant cancer cells within a tumor mass, or hyperresponsive immune cells within a seemingly homogeneous group. scRNA-seq is also ideal for examination of single cells where each cell is essentially phenotypically unique, such as the analysis of individual T lymphocytes expressing unique T-cell receptors, or neurons within the brain. scRNA-seq is also increasingly being used to trace lineage and developmental relationships between heterogeneous, yet related, cellular states in embryonal development, cancer, organ-specific epithelium differentiation and lymphocyte fate diversity.

Although variations and custom modifications abound in the published literature, a general workflow for scRNA-seq studies can be summarized as follows. The first step in conducting scRNA-seq is isolation of viable, single cells (or nuclei) from the experimental sample, e.g., cells grown in vitro, blood, tissue of interest. Current methods then rely on isolating/partitioning of these single cells or nuclei thereof together with barcoded oligonucleotides attached to beads into physically separate compartments/partitions (e.g., microwells) or into individual droplets within microfluidic devices (e.g., as discussed in greater detail below). For single-cell analysis each compartment usually comprises one cell and one bead, where oligonucleotides attached to the bead have the same unique bead-specific (cell-specific) barcode. Next, isolated individual cells are lysed to release mRNA molecules, which then hybridize with barcoded oligo dT primers attached to or released from the bead. Next, the resultant oligo[T]-primed mRNAs are converted to barcoded complementary DNA (cDNA) by a reverse transcriptase. Barcoded cDNAs derived from different cells are then mixed together and amplified for the follow-up expression analysis.

Depending on the scRNA-seq protocol, the reverse-transcription primers usually also have adaptor sequences for amplification step, unique molecular identifiers (UMIs) to mark unequivocally a single mRNA molecule, as well as barcode sequences to label the sequences coming from an individual cell. The tiny amounts of cDNA are then amplified by PCR-based methods. Then, amplified and barcoded/cDNAs are sequenced by NGS, using library preparation, sequencing methods and genome-alignment tools similar to those used for bulk samples.

An important step in single-cell analysis methods to date has been the isolation of one cell with one barcoded bead in a physically separated compartment prior to lysis of the cell. One cell-one bead compartmentalization allows one to perform enzymatic reactions or physical interaction between the RNA or DNA released from single cell with barcoded oligonucleotides without mixing or contamination by the nucleic acids of other cells in an experimental sample. The requirement for compartmentalization has been generally solved by two main approaches: droplet-based methods or physical isolation of one cell—one bead composition into microwell compartments and sealing of these microwells. Droplet-based platforms (for example, Chromium from 10× Genomics, ddSEQ from Bio-Rad Laboratories, InDrop from 1CellBio, and pEncapsulator or Nadia from Dolomite Bio/Blacktrace Holdings) are commercially available and most commonly used for single-cell genetic analysis. Unfortunately, droplet-based instruments generate one-cell-one bead compositions only in small number of droplets defined by probability of presence one cell and one bead in small volume of a water droplet depending on concentration of cells and beads in water solution at the stage of droplet formation. Droplet-based instruments could encapsulate thousands of single cells-beads compositions in individual partitions (emulsion droplets), each containing all the necessary reagents for cell lysis, reverse transcription, cellular barcoding and molecular tagging. This eliminates the need for single-cell isolation through cell sorting or other approaches that result in physically separated cells within microwells. The latter approach includes commercial platforms such as the BD Rhapsody, the ICELL8 Single-Cell System (Takara) or custom protocols that rely on flow cytometric sorting or random deposition of single cells and barcoded beads or barcoded oligonucleotides into wells of microplates usually in two sequential steps. The droplet-based methodologies have been widely adopted, and dominate the current landscape of scRNA-seq due to more simple protocol and possibility to scale-up the analysis from dozens to thousand cells.

The available compartmentalization methods have allowed substantial progress in the growing single-cell analysis field. Nevertheless, they suffer from several intrinsic limitations. Among these are:
Lack of selectivity of isolating specific cell type, cell properties (e.g. viable, activated, etc.) from starting biological sample. It would be desirable to identify subpopulations of interest within the single cell suspension that are the starting point for single-cell protocols. Similarly, it would be desirable to identify and only analyze viable cells, excluding the variable number of dead or damaged cells often present within experimental samples. At present this goal is difficult to achieve in most droplet-based protocols, although some work-arounds that add rather complicated additional steps have been reported (e.g., labeling of cells with bar-coded antibodies or sorting the desired population of cells prior the droplet-based analysis.
Empty or overcrowded droplets/wells. The compartmentalization of cells and barcoded beads into droplets or single wells based on statistical distribution beads and cells in solution results in a substantial number of empty (no cell, no bead or no cell-no bead) or overcrowded (2 or more cells with one bead or one cell with two or more beads) in compartments. Therefore, significant number of compartments can't be used for single-cell analysis as it should contain a single cell-single barcoded bead composition. Such empty and overcrowded droplets/wells result in a substantial waste of reagents, reduce the yield of usable data, and introduce undesirable complexity into the data analysis steps. Typical number of empty wells, cell doublets, etc. even in an optimized protocol is not less than 70%.
Instrumentation cost and operation. The sophisticated instrumentation for either droplet- or cell sorting-based scRNA-seq is expensive, and requires similarly expensive reagent kits and technical personnel for optimal operation. Compartmentalization as a common problem. A major source of the limitations of current methodologies is the need for strict physical compartmentalization of single cells, single barcoded bead with reagents and under conditions that allow to release and tagging of cellular nucleic acids (e.g. RNA) for subsequent processing.

The inventors of the present application have identified the following needs in single cell genetic analysis protocols, such as scRNA-seq protocols:
Simplicity. Compartment based workflows rely on complex microfluidic instrumentation whose sophistication requires substantial training for potential users. The expense also limits use of the technology to well-funded companies, institutions or core facilities. A simplified method that avoids the need for such instrumentation would allow adoption of scRNA-seq by many more laboratories.
Cell specificity/selection. For many applications, a specific subset of cells within tissue or biological fluid (e.g., blood) samples is of interest. The current compartmentalization-based approaches rely on post-analysis sorting in silico of subpopulations by their transcriptional profiles. This requires reagent use and data analysis for many cells that are a priori of no actual interest, thus increasing expense and time costs. Similarly, data obtained from dead or dying cells included in most droplet-based analyses have to be identified and filtered out using bioinformatic methods. A methodology that begins with sorting or identification of the (live) cells of interest would improve throughput and efficiency.
Compatibility with multiple gene expression protocols. A methodology that is platform-independent and can be adapted to various gene expression protocols would be desirable and allow many different users to make progress in the single cell field. Scaling-up analysis to hundred thousand-million cells. Many applications of single-cell profiling (e.g. whole embryo, genetic screen with effector libraries (sgRNA, shRNA, proteins, etc.) require cost-effective analysis of at least hundred thousand cells which is not practical using current instrumentation and available protocols.

### SUMMARY

In order to address the limitations of current compartmentalization-based protocols and address one or more of the identified needs discussed above, aspects of the present invention provide for compartment-free single cell genetic analysis protocols which do not require partitioning of cells into separate compartments. The compartment-free protocols described herein allow one to perform single-cell analysis in practically any scale (up to millions of cells) without any specialized instrumentation. Moreover, the compartment-free protocols described herein can be combined with phenotype-based conventional cell sorting instruments for analysis of specific cell fractions.

Compartment-free single cell genetic analysis methods are provided. Aspects of the methods include: (a) combining a cellular sample with a plurality of distinct barcoded beads comprising barcoded reverse primers under conditions sufficient to produce a liquid composition comprising a plurality of separated cell/barcoded bead complexes; (b) hybridizing template binding domains of barcoded reverse primers to template nucleic acids of the cells to produce primed template nucleic acids; and (c) subjecting the primed template nucleic acids to primer extension reaction conditions sufficient to produce barcoded nucleic acids, e.g., for subsequent amplification and analysis, such as by Next Generation Sequencing (NGS) protocols. Also provided are compositions that find use in practicing embodiments of the methods.

### BRIEF DESCRIPTION OF THE FIGURES

FIG. 1 provides a schematic of a scRNA-seq protocol according to an embodiment of the invention in which gene specific reverse primers are employed.

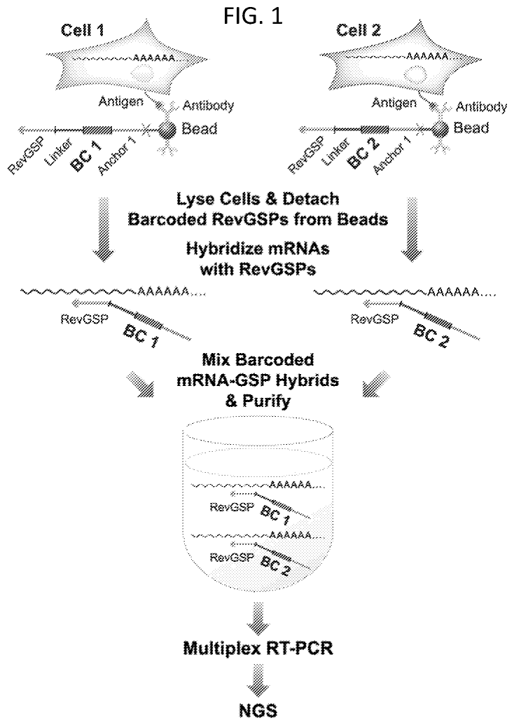

FIG. 2 provides a schematic of a scRNA-seq protocol according to an embodiment of the invention in which oligo-dT reverse primers are employed.

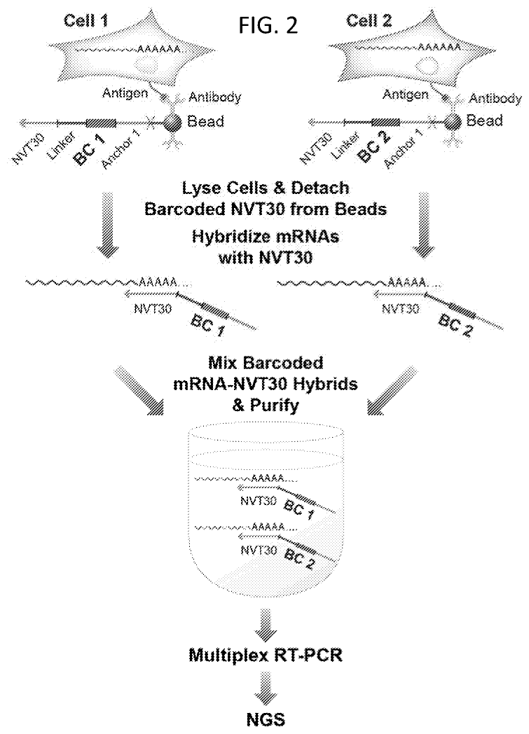

FIG. 3 provides a schematic of a scRNA-seq protocol that employs a stimulus responsive polymer with gene specific reverse primers, according to an embodiment of the invention.

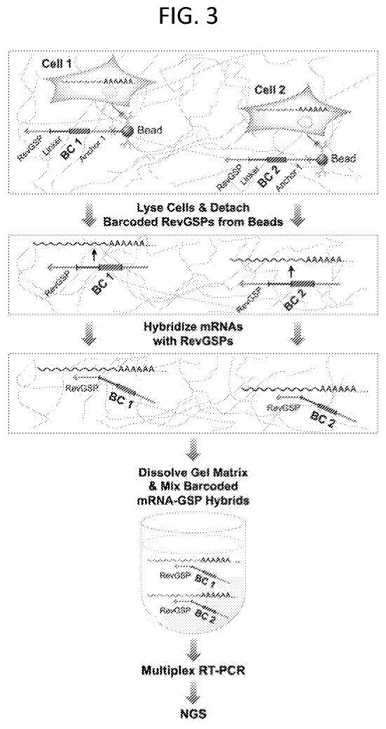

FIG. 4 provides a schematic of a scRNA-seq protocol that employs a stimulus responsive polymer with oligo-dT reverse primers, according to an embodiment of the invention.

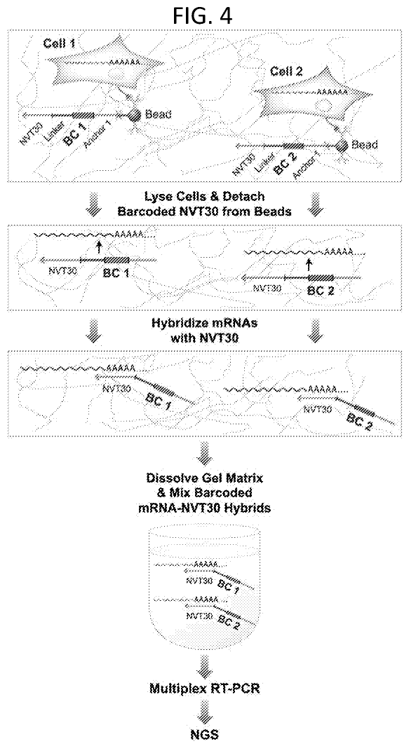

FIG. 5 provides a schematic of a scRNA-seq protocol that includes a sorting step, according to an embodiment of the invention.

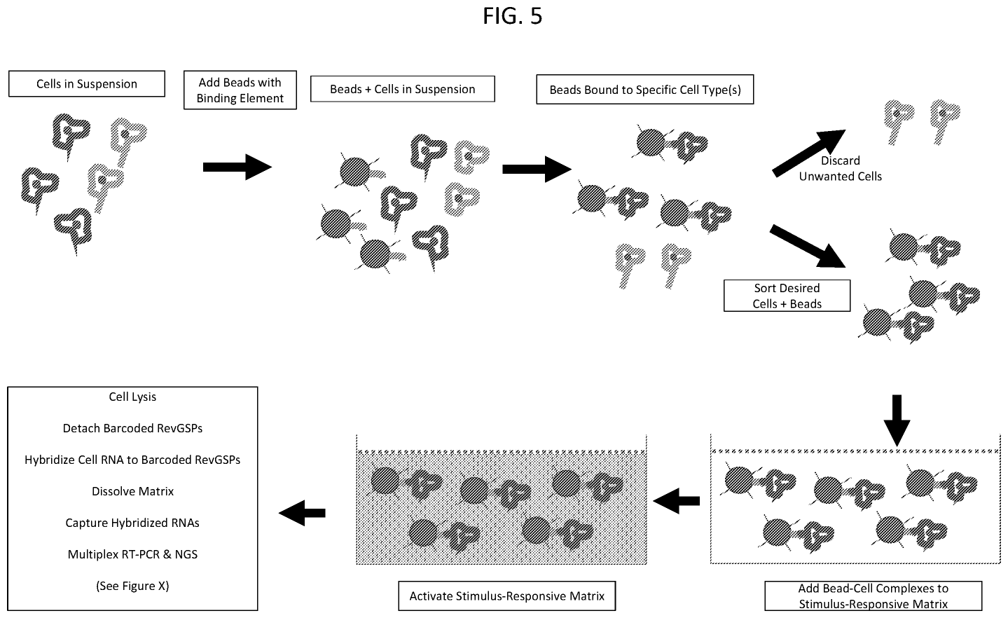

FIG. 6 provides a schematic of a scRNA-seq protocol in which cell/barcoded bead complexes are present on a solid support, according to an embodiment of the invention.

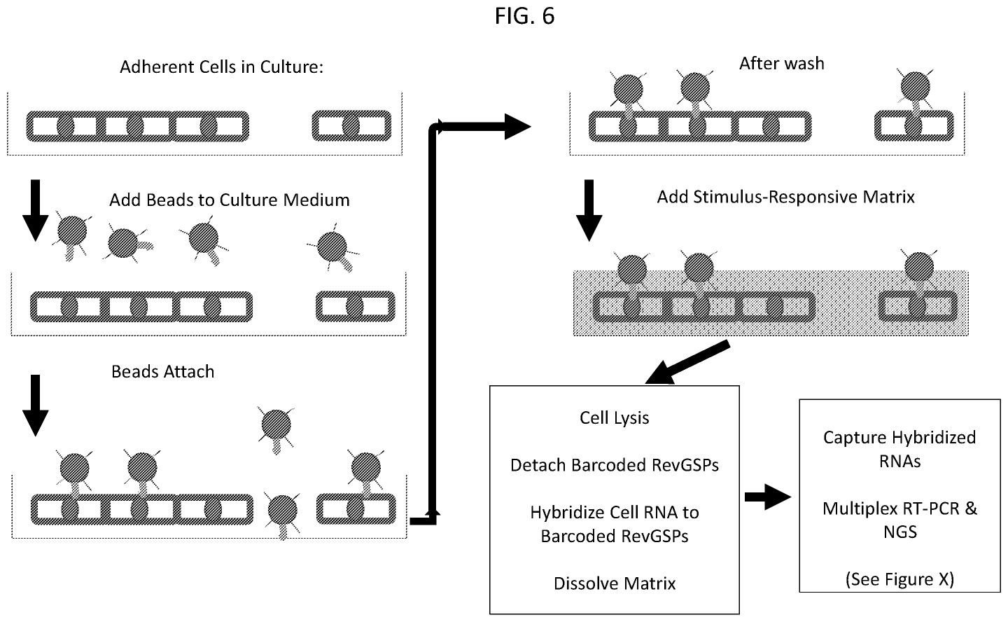

FIG. 7 provides a schematic diagram in which a cell sample that is first prepared from a single guide RNA (sgRNA) clonal barcode effector library (e.g., as described in U.S. Pat. Nos. 9,429,565 and 10,196,634 (the disclosures of which are herein incorporated by reference)) is analyzed by a scRNA-seq protocol in which cell/barcoded bead complexes are present on a solid support, according to an embodiment of the invention.

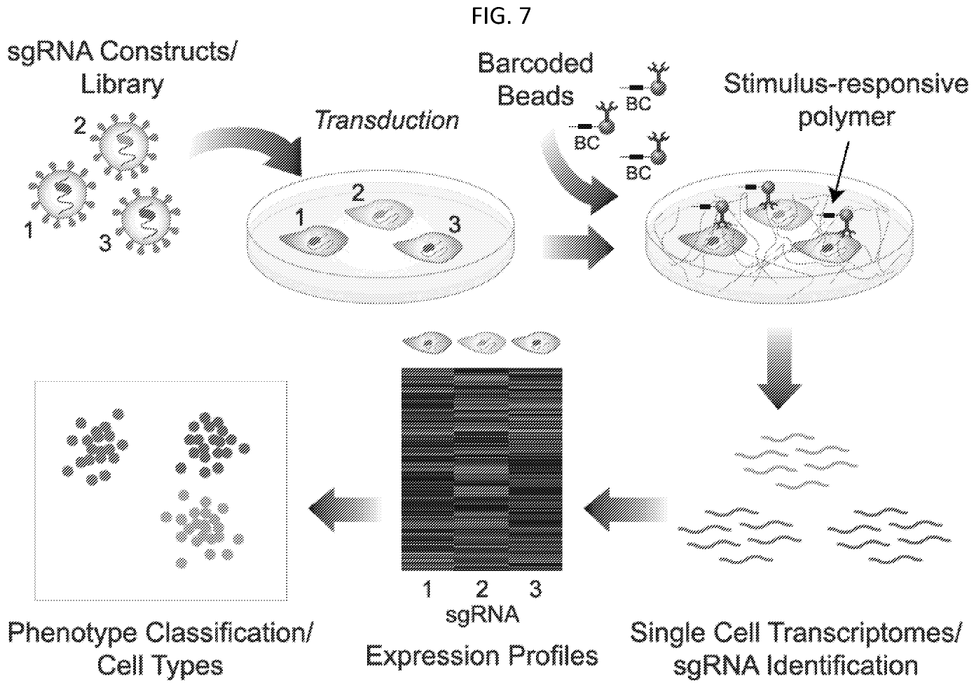

### DEFINITIONS

As used herein, the term “hybridization conditions” means conditions in which a primer, or other oligonucleotide, specifically hybridizes to a region of a target nucleic acid with which the primer or other oligonucleotide shares significant complementarity. Whether a primer specifically hybridizes to a target nucleic acid is determined by such factors as the degree of complementarity between the oligonucleotide and the target nucleic acid and the temperature at which the hybridization occurs, which may be informed by the melting temperature (Tm) of the primer. The melting temperature refers to the temperature at which half of the primer-target nucleic acid duplexes remain hybridized and half of the duplexes dissociate into single strands. The Tm of a duplex may be experimentally determined or predicted using the following formula Tm=81.5+16.6(log 10[Na+])+0.41 (fraction G+C)−(60/N), where N is the chain length and [Na+] is less than 1 M. See Sambrook and Russell (2001; Molecular Cloning: A Laboratory Manual, 3rd ed., Cold Spring Harbor Press, Cold Spring Harbor N.Y., Ch. 10). Other more advanced models that depend on various parameters may also be used to predict Tm of primer/target duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

The terms “complementary” and “complementarity” as used herein refer to a nucleotide sequence that base-pairs by non-covalent bonds to all or a region of a target nucleic acid (e.g., a protein-coding region of the gene). As described in a more detail below, a barcoded oligonucleotide primer may be complementary to, and therefore may hybridize to, a target nucleic acid and therefore form a primed target nucleic acid hybrid. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, a primer may be perfectly (i.e., 100%) complementary to the target nucleic acid, or the primer and the target nucleic acid may share some degree of complementarity which is less than perfect (e.g., 70%, 75%, 85%, 90%, 95%, 99%).

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

A “domain” refers to a stretch or length of a nucleic acid made up of a plurality of nucleotides, where the stretch or length provides a defined function to the nucleic acid. Examples of domains include primer binding or anchor domains, hybridization (template-binding or gene-specific primer) domains, barcode domains (such as source/sample barcode domains), unique molecular identifier (UMI) domains, Next Generation Sequencing (NGS) adaptor domains, NGS indexing domains, etc. In some instances, the terms “domain” and “region” may be used interchangeably, where the length of a given domain may vary, in some instances the length ranges from 2 to 100 nucleotides (nt), such as 5 to 50 nt, e.g., 5 to 30 nt.

“Barcoded beads” (or barcoded oligonucleotide beads) are polymeric, hydrogel, glass, metal or composite particles with covalently or non-covalently attached barcoded oligonucleotides. In some embodiments, all oligonucleotides attached to a given bead have the same barcode domain (which barcode domain is specific for the bead but is different from that found in oligonucleotides of any other beads being used in a given assay), and the same anchor domain. Furthermore, the barcoded oligonucleotides may include a template-binding domains or gene-specific primer domains which could be a single universal sequence (e.g. oligo dT primer) or plurality of different sequences, e.g., gene-specific primer compositions complementary to the target nucleic acid sequences, e.g., as described in greater detail below.

By “primer extension product composition” is meant a nucleic acid composition that includes nucleic acids that are primer extension products. Primer extension products are deoxyribonucleic acids that include a primer domain at the 5′ end covalently bonded to a synthesized domain at the 3′ end, which synthesized domain is a domain of base residues added by a polymerase mediated reaction to the 3′ end of the primer domain. The synthesized domain is a sequence that is dictated by a template nucleic acid to which the primer domain is hybridized and formed primed template nucleic acid compositions during production of the primer extension product. Primer extension product compositions may include double stranded nucleic acids that include a template nucleic acid strand complementary to a primer extension product strand, e.g., as described above. The length of the primer extension products and/or double stranded nucleic acids that incorporate the same in the primer extension product compositions may vary, wherein in some instances the nucleic acids have a length ranging from 50 to 1000 nt, such as 60 to 400 nt and including 70 to 250 nt. The number of distinct nucleic acids that differ from each other by sequence in the primer extension product compositions produced via methods of the invention may also vary, ranging in some instances from 10 to 50,000, such as 100 to 20,000 and including 1,000 to 10,000, 10,000 to 20,000, 15,000 to 20,000 and 15,000 to 19,000.

“Barcode” is the domain in the oligonucleotide attached to bead which is specific to that individual bead. A given barcode domain may vary length, and in some instances ranges from 6 to 100 nt, such as from 10-50 nt and including from 12-30 nt. Barcode domains may be synthesized by conventional combinatorial or split-pool synthesis protocols using bead-oligonucleotide conjugates, wherein an initial oligonucleotide (e.g., attached the beads) includes any common domain(s), such as primer binding or anchor domains. In a split-pool strategy, the plurality of bead-oligonucleotide conjugates is split into several separate compartments (e.g. 4) and unique nucleotides or stretches of nucleotides are attached to the 3′-ends of oligonucleotides, e.g., A in compartment 1, G in compartment 2, C in compartment 3 and T in compartment 4. In the next synthesis cycle, all the beads pooled together, split again into several compartments and the next barcode-specific sequence is added to the each subpool oligonucleotide. The split-pool synthesis usually continues until each bead carries a unique barcoded oligonucleotide specific for that bead. Synthesis of barcoded beads can be performed by conventional phosphoramidate chemistry or by enzymatic addition of barcoded sub-domains using any conventional protocol, e.g., based on ligation or primer extension reaction.

“Barcoded bead/cell complex” means a composition that includes at least one cell or component thereof (e.g., nucleus) and one barcoded bead, where the cell and bead may be attached to each other, e.g., via a specific binding pair interaction such as cellular binding moiety attached to the bead. In some embodiments, e.g., to study cell-to-cell interactions, the complex could include two or more cells and one bead. In another application the complex could be used with one cell attached to two beads each with unique barcode. Barcoded beads and cells could be attached to each other through covalent or non-covalent bonds. Covalent bonds could be formed by using cross-linking reagents. Non-covalent interaction between barcoded beads and cells can be achieved by attaching a cell interacting/binding moiety to the bead, where examples of cell binding moieties include antibodies, aptamers, lipid molecules, etc., which cell interacting moieties could interact with and bind to moieties present in the cell surface. Cell interacting moieties may be non-specific with respect to cell type (e.g., a lipid cell interacting moieties that interacts with a cell membrane or a moiety interacting with cell surface based on electrostatic, hydrophobic, etc., interactions), specific for a given cell type (e.g., an antibody recognizing cell-type specific antigen), or a combination of both. The cell/barcoded bead complex interaction may be sufficiently stable to allow for separation of complexes from each other, e.g., by FACS, dilution, binding to a surface, etc.

“Separation of cell/barcoded bead complexes” refers to the separation in space of different cell/barcoded bead complexes at the distance which minimizes, if not prevents, interaction between cell-derived nucleic acids and barcoded oligonucleotides derived from different complexes. For example, in some embodiments the distance between any two cell/barcoded bead complexes is more than diffusion distance of barcoded oligonucleotides involved in hybridization with cell-derived template nucleic acid (RNA or DNA). For example, if the diffusion distance of barcoded oligonucleotides under hybridization conditions is 200 microns, the cell/bead complexes separated from each other for the distance, e.g., 500-1000 microns, will be at the distance which prevents hybridization of barcoded oligonucleotides from one complex to nucleic acids derived from other complexes. The distance between different cell/bead complexes may vary, and in some instances ranges from 50 microns to 100,000 microns, such as from 100 microns to 10,000 microns, including 200 microns to 3,000 microns, such as 300 microns to 2,000 microns. In some instances, the media which separates cell/barcoded bead complexes from each other is an aqueous buffered solution that includes polymeric molecules or gel which reduce diffusion speed of molecules and minimize the movement of cell/bead complexes. Some embodiments of the present invention employ stimuli-responsive polymers, e.g., hydrogels, which are liquid at normal physiological conditions, e.g., conditions which allow separation of cell/bead complexes from each other in space. After applying a stimulus (e.g., heat, UV-light, pH, etc.) the polymer is solidified, e.g., to form a gel which prevents diffusion of cell-derived nucleic acids and barcoded oligonucleotides (e.g., detached from the beads). Examples of stimulus-responsive polymers are methylcellulose, poly(N-isopropylacrylamide), etc.

“Compartment-free” means separation of cell/bead complexes from each other in space as an alternative to current compartment-based protocols based on separation of each cell-bead composition from each other in different compartments by walls (e.g., “walls” created by oil interface for microdroplets or microwells). In contrast to the present invention, in compartment-based protocols, walls (provided by physical barriers or an immiscible fluid barrier) surround the cell/bead composition from all sides and prevent diffusion and interaction of barcoded oligonucleotides and nucleic acids located in different compartments. The current invention discloses that if cell-bead complexes are separated from each other the walls are not necessary. The cell-bead complexes can be separated from each other in one, two or three dimensions. Examples of separation in one dimension could be capillaries with diameter of the lumen close to the size of cell-bead complex. Cell-bead complexes could be also attached or deposited at a distance from each other on a solid surface, e.g., plastic, glass, metal, etc. Cell-bead complexes could be attached to a cell surface through cell or bead. In order to provide a uniform, similar distance between different cell/bead complexes attached to the surface, the surface may have small areas which could bind the cell-bead complexes (similar or smaller than the size of cell-bead complex, e.g., from 1-50 microns, 2-40 microns, 5-20 microns) which are separated from each other by areas which cannot bind the cell/bead complexes. Cell/bead binding surfaces may be plastic or glass surfaces covered by cell-binding polymers (fibronectin, antibodies, aptamers, collagen, etc.), chemically-modified hydrophilic surfaces which could non-specifically bind cells (or beads) though electrostatic, hydrophobic, etc., interactions, and the like. Examples of surface areas which are not intended to bind cell/bead complexes include hydrophobic surfaces, some elevations (e.g., in the form of walls) which separate cell-bead complexes from each other but are still open from the top for delivery cell-bead complexes and reagent for follow-up cell lysis and hybridization steps. In some instances, cell-bead complexes are just separated from each other in all 3 dimensions in a volume of solution, e.g., methylcellulose liquid polymer in physiological buffer. Separation of cell-bead complexes in volume of solution allows one to achieve the most efficient separation of large numbers of cells.

“Cellular sample” means a liquid composition of plurality of cells, e.g., eukaryotic cells, or components thereof, e.g., nuclei. A cellular sample may be obtained from a biological source, such as normal or diseased tissue, biological fluids (blood, saliva, lymphatic liquid, etc.), cell fractions or cells grown in vitro, ex vivo or in vivo. A cellular sample obtained from biological source could be used directly or treated with physical, biological or chemical entities (e.g., anti-cancer drugs) prior the use in the single cell assay. In some instances, a cellular sample is a plurality of single cells isolated from a biological source by dissociation of multicellular structures or cell aggregates, e.g., using any convenient protocol. In some embodiments, a cell sample may include cellular structural components, such as nucleus, cytoplasm, mitochondria derived from single cell and having DNA or RNA component, etc. In some embodiments a cell sample includes two or more cells (e.g., organoids, cluster of cells) which are attached together based on natural cell-cell interactions necessary to perform a biological function (e.g., stroma-epithelial, immune-cancer, etc. cell-cell interaction). In some embodiment of the current invention, the cellular sample is made up of a plurality of cells genetically modified by delivering genetic effector constructs in target cells by conventional protocols, e.g., viral transduction. As disclosed U.S. Pat. Nos. 9,429,565 and 10,196,634 (the disclosures of which are herein incorporated by reference), the effectors comprise a wide range of molecules including sgRNA, shRNA, aptamers, antisense RNA, microRNAs, peptide, native or modified proteins, etc., which effectors may be expressed in the target cells and change the cells' genotype and/or phenotype. The expression of effector molecules may change expression or regulation of target genes (e.g., drug targets), express modified version of target proteins (e.g., oncogenic mutated proteins), etc. The expression of effector molecules is a key technology for genetic screen and studying gene functions, e.g., discovery of novel drug targets for development of novel drugs. The effector constructs may also include clonal barcodes which allows for labelling each genetically modified cell and its progeny with cell-specific barcodes. Clonal barcodes, e.g., as described in the above patents, may be used for labelling both genomic DNA and expressed effector RNAs in individual cells, therefore providing additional (bead derived) barcode for cell tracing. Clonal barcodes are further described in U.S. patent application Serial No. Single cell analysis using protocols disclosed in the current invention with genetically modified cells allows one to link expression profile with effector molecules in each specific cellular clone.

### DETAILED DESCRIPTION

Compartment-free single cell genetic analysis methods are provided. Aspects of the methods include: (a) combining a cellular sample with a plurality of distinct barcoded beads comprising barcoded reverse primers under conditions sufficient to produce a liquid composition comprising a plurality of separated cell/barcoded bead complexes; (b) hybridizing template binding domains of barcoded reverse primers to template nucleic acids of the cells to produce primed template nucleic acids; and (c) subjecting the primed template nucleic acids to primer extension reaction conditions sufficient to produce barcoded nucleic acids, e.g., for subsequent amplification and analysis, such as by Next Generation Sequencing (NGS) protocols. Also provided are compositions that find use in practicing embodiments of the methods. The methods and compositions described herein find use in a variety of different applications, including single-cell expression profiling of RNAs and proteins, mutation and epigenetic analysis in genomic DNA, gene function analysis, drug target, small molecule and biologics screening applications.

Before the present invention is described in greater details, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing various aspects of the invention, embodiments of various methods will be discussed first in greater detail, followed by a review of various applications in which the methods find use as well as kits that find use in various embodiments of the invention.

### Methods

As summarized above, compartment-free methods of preparing barcoded nucleic acids are provided. As the methods are compartment-free, they are performed in the absence of partitions or compartments, e.g., as described above. Accordingly, the methods are not performed in sealed microwells or in aqueous droplets of an emulsion. Therefore, in embodiments of the methods, physical walls surrounding cell/bead complexes from all sides are not present. Furthermore, in embodiments of the methods, cell/bead complexes are not present in an aqueous droplet present in an immiscible liquid, e.g., oil. As such, embodiments of the methods do not employ microwell plates or droplet producing microfluidic devices.

### Production of Cell/Barcoded Bead Complexes

Embodiments of the methods include combining a cellular sample, e.g., as described above, with a plurality of distinct barcoded beads, e.g., as described above, under conditions sufficient to produce a liquid composition comprising a plurality of separated cell/barcoded bead complexes. The cell/barcoded bead complexes may include a single cell or component thereof (e.g., nucleus) and a single barcoded bead, or two or more cells (or components thereof) and a single barcoded bead, or a single cell (or component thereof) and two or more barcoded beads. Of interest in some embodiments are cell/barcoded bead complexes that include a single cell or single nucleus and a single barcoded bead. In embodiments of this step of the methods, the cell/barcoded complexes are separated from each other in liquid composition, such as an aqueous liquid composition, such that cell/barcoded bead complexes in the liquid composition do not touch each other.

As such, barcoded beads may interact with a cell (or nuclei isolated from cells) population to produce cell/barcoded bead complexes made up of single cell-single bead pairs, or cell/barcoded bead complexes comprised of a single barcoded bead and two or more cells or a single cell bound to two or more barcoded beads. In some instances, single cell-single bead complexes are of interest since they provide the specific genetic analysis of a cell population at single cell resolution as identified by the barcoding sequences of the barcoded reverse primers. The generation of single cell/barcoded bead complexes with low percentage of multiple bead or multiple cell complexes may be achieved by optimizing the ratio between cells and barcoded beads, e.g., using an excess of beads from number of cells. The single cell/bead complexes may also be enriched from multiple cell/beads complexes by any conventional separation protocols, including filtration through pores, centrifugation, electrophoresis, etc. The use of flow cytometric sorting allows isolation of single cell-single bead pair complexes for use in compartment-free assays as herein. In some instances, small numbers of complexes comprised of one cell with multiple beads or one bead with multiple cells may enter the analytical workflow. In the case of one cell binding multiple beads, the resultant genetic profile of the cell will be attributed to two or more cells. This is unlikely to skew results significantly, based on the low frequency of these events and the preservation of signature of the cell, albeit now divided by two or more bead-specific barcodes into two or more separate but similar profiles within the population of cells under study. For the case of one bead binding multiple cells, this may lead to confounding results e.g., in transcriptional analysis, since this may attribute incorrectly high levels of expression of certain genes to a single bead-specific barcode since the single bead's oligonucleotides will now be capturing the RNA from two or more cells. The magnitude of one-bead-multiple cell complexes can be assessed using cells labelled (e.g., by viral transduction with barcoded genetic constructs) with UMI RNAs extension products derived from two or more cells and labelled with a single bead's barcoded oligonucleotides. The two cell-bead complex suspensions isolated by sorting can be mixed and placed into the compartment-free polymer matrix, e.g., as described herein. In the absence of any crosstalk, all of cell type 1 will produce sequences tagged with sample barcodes from bead population 1, and all of cell type 2 will produce sequences tagged with sample barcodes from bead population 2. The proportion of cross-talk, if any, can be quantitated by the number of cell type 1 RNAs identified by barcodes from bead population 2 and vice versa.

In some applications, e.g., those designed for analysis of cell-interaction, the binding of one bead to two or more cells is beneficial as such allows one to identify and profile the cells which are naturally close and interact with each other in vivo. One bead-two cell complexes may be isolated by FACS or other suitable technology from biological sample, e.g., tissue sample partially disintegrated to the level of 1-5 cell aggregates.

In compartment-free protocols of the invention, detached barcoded reverse primers from one cell/barcoded bead complex may diffuse to the proximity of a different cell/barcoded bead complex, and subsequently hybridize to target RNA of the second cell/barcoded bead complex, thereby confounding the single cell specificity of the genetic analysis. This can be evaluated by mixing experiments using two distinct cell types transfected respectively with one of two different cell identifier barcodes, bound respectively to two bead populations with distinct barcode sets. In order to avoid crosstalk between different cell-bead complexes, embodiments of the current invention disclose that bead-cell complexes should be separated from each at the distance which exceeds diffusion limit of barcoded oligonucleotides (if they are detached from beads), and nucleic acids (RNA or DNA) interacting with each other during the time course of the assay. Diffusion distance depends on many factors, but the most important is size of molecules. In embodiments of methods described herein, the barcoded reverse primers detached from beads are defined the diffusion distance as both RNA and DNA molecules have significantly (at least 10-1,000-fold) higher molecular mass. Diffusion distance of the oligonucleotides can be measured experimentally as disclosed in the example section or calculated based on other approaches known in the art. Based on experimental or theoretical calculations, the concentration of cell-bead complexes (e.g., distance between complexes) could be adjusted accordingly to minimize the cross-talk between different cell-barcoded bead complexes. For example, if diffusion distance of barcoded reverse primers is 100 microns (under hybridization conditions used in a given protocol) the optimal mean distance between cell-barcoded bead complexes may be chosen to be 100 microns or longer, such as 200 microns or longer and including 500 microns or longer. In methods of the invention, the cell/barcoded bead complexes may be suspended in the liquid composition or present on a support surface in the liquid composition. While the distance separating the cell/barcoded bead complexes may vary, where in some instances the distance separating the cell/barcoded bead complexes is 100 microns or longer, such as 500 microns or longer, including 1,000 microns or longer. In some instances, the distance separating cell/barcoded bead complexes ranges from 100 microns to 100,000 microns, such as from 200 microns to 10,000 microns, including 300 microns to 3,000 microns, such as 500 microns to 2,000 microns.

Barcoded beads employed in methods of the invention may vary, and include a bead component having present on the surface thereof barcoded reverse primers. The bead component can be made of a polymeric material (e.g., polystyrene, acrylamide, hydrogel, etc.) but may be made of other materials as well (e.g., glass, metal, magnetic bead with iron core surrounded by polymeric shell, etc.). The beads can be non-modified or chemically modified at the surface (e.g., sulfated, amidated, carboxylated, etc.) to provide for binding to oligonucleotides or to use as a starting support for oligonucleotide synthesis. The size of the beads may vary, where in some instances the diameter of the beads ranges from 1 to 1,000 microns, such as 2 to 500 microns, including 3 to 200 microns, e.g., 5-50 microns, e.g., 10-30 microns. In some instances, the size of the bead is selected to correspond to the size of the cellular component of the cell/barded bead complexes to be produced in a given protocol. For example, where a given protocol is a single cell analysis protocol, the barcoded beads may have a diameter ranging from 1 to 30 microns. For analysis of cell clusters (e.g. organoids), the barcoded beads may have a diameter ranging from 10 to 100 microns. The shape of the beads may also vary, ranging from spherical structure to other shapes (e.g., cylinder, cube, irregular, etc.). The bead components may be non-porous or porous, e.g., where pores may be provided to impart a higher surface density of immobilized molecules. The beads may also be covered by a polymeric layer to increase the amount of attached barcoded oligonucleotides.

The barcoded beads employed in methods of the invention include beads with a plurality of barcoded reverse primers attached thereto. While the number of barcoded reverse primers attached to any given bead may vary, in some instances the number of barcoded reverse primers attached to any given bead is 100 or more and 10¹² or less, and in some instances the number ranges from 10⁵ to 10¹², such as 10⁶ to 10¹², including 10⁷ to 10¹¹ e.g., 10⁸ to 10¹⁰ barcoded reverse primers. In some instances, all barcoded reverse primers attached to a given bead have the same barcode domain, such that they share a common barcode domain. In other embodiments one bead could carry two or more barcode domains among the barcoded reverse primers attached thereto. In embodiments of the methods, within a given plurality of barcoded beads, the majority of, if not all of, the barcoded beads have different barcodes from each other. For example, if a given protocol is designed to profile 10,000 cells and uses 100,000 barcoded beads, the 100,000 barcodes attached to the 100,000 barcoded beads are significantly different from each other, such that at least 95%, such as 99% and including 99.9% of beads have different barcodes.

In embodiments, the barcoded reverse primers include a number of different domains, which domains may include a template binding domain, a barcode domain and an anchor domain, wherein in some instances the order these domains from the 5′ end to the 3′ end is the anchor domain, the barcode domain and the template binding domain.

Anchor domains are domains that are employed in nucleic acid amplification steps of the methods, such as polymerase chain reaction (PCR), where anchor domains serve as primer binding sites for the primers employed in such amplification steps. Where the amplification employed is PCR, the anchor domains may also be referred to as PCR primer binding domains. The length of the anchor domains may vary, as desired. In some instances, anchor domains range in length from 10 to 50 nt, such as 15 to 30 nt, e.g., 18 to 28, including 18 to 26 nt. Where desired, the anchor domains may include PCR suppression sequences. PCR suppression sequences are sequences configured to suppress the formation of non-target DNA amplification products (e.g., primer dimers) during PCR amplification reactions, e.g., via the production of pan-like structures. Such sequences, when present, may vary in length, ranging in some instances from 5 to 25 nt, such as 7 to 21, including 7 to 20 nt. PCR suppression sequences of interest include, but are not limited to, those sequences described in U.S. Pat. No. 5,565,340; the disclosure of which is herein incorporated by reference. An example of forward and reverse anchor domains that include PCR suppression sequences are: AGCACCGACCAGCAGACA (SEQ ID NO:01) and AGCACCGACCAGCACAGA (SEQ ID NO:02).

Barcoded reverse primers also include a barcode domain. A barcode domain is a domain that denotes, i.e., indicates or provides, information about (such that it may be used to determine), the specific bead and therefore cell associated therewith in a given cell/barcoded bead complex, from which primed template nucleic acids are produced. Barcode domains include unique, specific sequences. While the length of a given barcode domain may vary, in some instances the length ranges from 6 to 30 nt, such as 8 to 20 nt, and including 12 to 18 nt.

Also present in the barcoded reverse primers are template binding domains. The template binding domain may vary depending on the particular assay. In some instances, the template binding domain is a consensus sequence(s) (e.g. oligo dT, template switching oligonucleotide (TSO, such as SMART® TSO Takara Bio USA), oligonucleotide specific to any specific genomic DNA or RNA sequence(s)) capable of binding to plurality (e.g. all mRNAs with polyA tail, template extended products, repetitive elements or homologous genes, etc.) or individual target template sequences (e.g. barcode integration, clonal barcode sequence, mutated genomic DNA, etc., sites). In yet other instances, the template-binding domains of the barcoded reverse primers of a given plurality of barcoded bead may be gene-specific template binding domains such that the plurality of barcoded beads including a population of reverse gene-specific primers. While the number of distinct primers in a given set may vary, as desired, in some instances the number of primers in a given set is 10 or more, such as 20 or more, 30 or more, 40 or more, 50 or more, 60 or more, 70 or more, 80 or more, 90 or more, 100 or more, 125 or more, 250 or more, 500 or more, including 1000 or more, 200 or more, 5000 or more, 8000 or more, 10,000 or more 15,000 or more, 18,000 or more and 20,000 or more. In some instances, the number of gene specific primers that is present in the set is 25,000 or less, such as 20,000 or less. As such, in some embodiments the number of gene specifics in the set that is employed in the methods ranges from 10 to 25,000, such as 50 to 20,000, including 1,000 to 10,000, e.g., 2,500 to 8,500, and 10,000 to 20,000, e.g., 15,000 to 19,000. Gene specific reverse primers include gene specific domains, where these gene specific domains may be experimentally validated as suitable for use in a multiplex amplification assay. By “experimentally validated as suitable for use in a multiplex amplification assay” is meant that primers for each target gene in a given set has been experimentally tested in a multiplex amplification assay, such as described in United States Published Patent Application Nos. 20160376664 and 20180245164, the disclosures of which are herein incorporated by reference. To control efficiency and specificity of primer hybridization and the subject extension step, the length of the gene specific domain of the gene specific primer may vary. In some instances, the length ranges from 10 to 120 nt, such as 15 to 75 nt, e.g., 16 to 50 nt, such as 18 to 40 nt, including 20 to 30 nt or 25 to 40 nt. The gene specific domain primer may vary length. In some instances, the length the gene specific domain in the reverse primers ranges from 25 to 80 nt, such as 30 to 70 nt, including 30 to 40 nt. As the gene specific primers are barcoded and may include additional domains, e.g., anchor domains, etc., in some embodiments the primers in length from 10 to 150 nt, such as 10 to 100 nt, including 10 to 75 nt, such as from 15 to 60 nt, including from 24 to 45 nt. Where desired, the gene specific primers may be GCA- and/or GCT-rich. By GCA- and/or GCT-rich is meant that the gene-specific primer domain has a substantial portion of G, C, A- and/or G, C, T nucleotides. While the number of such nucleotides in a gene specific primer domain may vary, in some instance the number of such sequences ranges from 75% to 100%, such as 85% to 100%. As the gene specific primer domains of such embodiments are GCA- and/or GCT-rich, the GC content of the gene specific primer domains is also high. While the GC content may vary, in some instances the GC content ranges from 40 to 90%, such as 45 to 85%, including 50 to 85%, e.g., 50 to 80%. Depending on the specific application for which the set is configured, the set of gene specific primers may be configured to target a wide range of mammalian genes, genetically modified genes or artificial or recombinant sequences (e.g. barcodes, genes, effector constructs) introduced in the cells, and pathogenic genes from a wide range of pathogenic organisms, such as viruses, bacteria, fungi, etc. which could be present in the human or mammalian bodies. Of interest in certain applications are human, mammalian species commonly used as a model organisms to study human diseases, such as mouse, rat, or monkey, and pathogenic organisms associated with human diseases. To be analyzed in accordance with embodiments of the invention, the targeted genes may be present in the mammalian cells or fluids. In some embodiments, the targeted genes are may be protein coding, or may express non-coding RNAs, micro RNAs, mitochondrial RNAs, regulatory RNAs, etc. In some instances, the set of genes selected is genome-wide, such that it covers all genes present in the genome of an organism. In other embodiments, the genes are selected from the genes that could be transcribed or expressed in the organism and present in the biological samples in the form of RNA. The genome-wide set of genes specific for human, model and pathogenic organisms is of special interest in some instances and may be used to develop a set of genome-wide targeted RNA expression assays based on the disclosed multiplex PCR assay. Genome-wide sets of primers may vary in number, and in some instances are configured to assay 18,000 or more, such as 20,000 or more and 25,000 or more, such as 30,000 or more genes. Additional sets of PCR primers may be configured based on a genome-wide set of genes from a wide range of viral, bacterial and eukaryotic pathogenic organisms. In another embodiment, the gene specific primers may be configured to produce primer extension products from a subset of specific genes selected from the genome-wide set of genes. Examples of sets of reverse gene-specific primers and their use in single cell genetic analysis applications is disclosed in U.S. patent application Ser. Nos. 15/133,184 and 16/543,211, the disclosures of which sets of gene specific reverse primers are incorporated herein by reference.

In addition to the above domains, the barcoded reverse primers of the barcoded beads may, where desired, include one or more additional domains. One type of additional domain that may be included is a unique molecular index (UMI) domain. UMI domains have sequences configured for labeling of each RNA molecule in a plurality of RNA molecules (and extended cDNA product) present in a hybridization mix with different molecule-specific indexes. UMI domains are stretches of random or semi-random nucleotides. While the lengths of UMI domains may vary, in some instances the length of a given UMI domain ranges from 8 to 20 nt, which in a given assay provides for complexity of different unique sequences of 10,000 or more different UMIs. In some instances, using at least 10,000 unique indexes is sufficient to label each template molecule present in one sample with a unique index, i.e., UMI. By analyzing the number of the indexes, e.g., via NGS, the number of each unique template molecules employed in multiplex PCR assay can be calculated. In some instances, when present the UMI domain may be combined with the barcode domain, e.g., where the UMI nucleotides are interspersed with the barcode nucleotides in a BUMI domain, e.g., as described in United States Patent Application Publication No. US20150072344, the disclosure of which is herein incorporated by reference.

In addition, barcoded reverse primers may include one or more linker domains. Linker domains are domains that link other domains together, e.g., barcode and template binding domains. While the length of a given linker domain may vary, in some instances the length ranges from 5 to 30 nt, such as 10 to 25 nt, including 12 to 20 nt. There are no special requirements for nucleotide composition or sequence of the linker domain, but in some instances the linker domain is selected with GC-content in the range 50% to 80% without significant secondary structure within the domain or with other domains present in the oligonucleotide.

The barcoded reverse primers, e.g., as described above, may be attached to the beads by non-covalent or covalent bonds. In some instances, the barcoded reverse primers are covalently attached to the beads, e.g., through a suitable linker. While any convenient linker may be employed, in some instances the linker is a cleavable linker, such as a photocleavable linker, a chemically cleavable linker, a thermosensitive linker and the like, which cleavable linkers allow for the release of barcoded oligonucleotides or barcoded extended DNA fragments from beads when desired. Such linkers include labile moieties, such as light labile moieties, chemical/enzymatic labile moieties, thermal-labile moieties etc., where examples of such moieties are disclosed Published United States Patent Application Publication No. US 2019-0112648 A1; the disclosure of which moieties and linkers including the same is herein incorporated by reference.

Examples of cleavable linkers that may be employed include, but are not limited to, thermal-labile linkers, enzymatically-labile linkers, light-labile linkers, etc. In some instances, the linker is a thermal labile linker that includes a thermally-labile blocking moiety. A thermally-labile blocking moiety is a moiety that may be cleaved when the temperature is raised above a certain threshold value to release barcoded primer from bead. While the threshold value may vary, in some instances the threshold value is 60° C. or higher, such as 75° C. or higher, including 90° C. or higher. Examples of thermally labile moieties that may be employed in accordance with the invention include, but are not limited to, those described in U.S. Pat. Nos. 8,133,669 and 8,361,753; the disclosures of which are herein incorporated by reference. In some instances, the thermally labile blocking moiety is a 3′ blocking moiety, such as but not limited to: O-phenoxyacetyl; O-methoxyacetyl; O-acetyl; O-(p-toluene)sulfonate; O-phosphate; O-nitrate; O-[4-methoxy]-tetrahydrothiopyranyl; O-tetrahydrothiopyranyl; O-[5-methyl]-tetrahydrofuranyl; O-[2-methyl,4-methoxy]-tetrahydropyranyl; O-[5-methyl]-tetrahydropyranyl; and O-tetrahydrothiofuranyl. In some instances, the linker is an enzymatically-labile linker. An enzymatically-labile linker includes a moiety that may be cleaved by exposing the linker to a suitable enzyme that cleaves the moiety. Examples of enzymatically-labile moieties of interest include those having a linkage group cleavable by a hydrolase enzyme. Examples of hydrolase enzymes of interest include, but are not limited to: esterases, phosphatases, peptidases, penicillin amidases, glycosidases and phosphorylases, kinases, etc. Hydrolase susceptible linkages and hydrolase enzymes are further described in U.S. Patent Application Publication No. 20050164182 and U.S. Pat. No. 7,078,499; the disclosures of which are herein incorporated by reference.

In some instances, the linker is a chemically-labile linker that includes a chemically-labile moiety. A chemically-labile is a moiety that may be cleaved by exposing the linker to a chemical agent that cleaves the moiety. The chemically-labile moiety may be reactive with the functional group of a chemical agent (e.g., an azido-containing modifiable group that is reactive with an alkynyl-containing reagent or a phosphine reagent, or vice versa, or a disulfide that is reactive with a reducing agent such as tris(2-carboxyethyl)phosphine (TCEP) or DTT). A variety of functional group chemistries and chemical agent stimuli suitable for modifying them may be utilized in the subject methods. Functional group chemistries and chemical agents of interest include, but are not limited to, click chemistry groups and reagents (e.g., as described by Sharpless et al., (2001), “Click Chemistry: Diverse Chemical Function from a Few Good Reactions”, Angewandte Chemie International Edition 40 (11): 2004-2021), Staudinger ligation groups and reagents (e.g., as described by Bertozzi et al., (2000), “Cell Surface Engineering by a Modified Staudinger Reaction”, Science 287 (5460): 2007), and other bioconjugation groups and reagents (e.g., as described by Hermanson, Bioconjugate Techniques, Second Edition, Academic Press, 2008). In certain embodiments, the chemically-labile blocking moiety includes a functional group selected from an azido, a phosphine (e.g., a triaryl phosphine or a trialkyl phosphine or mixtures thereof), a dithiol, an active ester, an alkynyl, a protected amino, a protected hydroxy, a protected thiol, a hydrazine, and a disulfide.

In some instances, the cleavable linker is a light-labile linker that includes a light-labile moiety, which is a moiety that may be cleaved by exposing the linker to light at a wavelength that cleaves the moiety from the linker. Examples of light-labile moieties of interest include cleavable by light of a certain wavelength that cleaves a photocleavable group in the linkage group. Any convenient photocleavable groups may find use. Cleavable groups and linkers may include photocleavable groups comprising covalent bonds that break upon exposure to light of a certain wavelength. Suitable photocleavable groups and linkers for use in the subject MCIPs include ortho-nitrobenzyl-based linkers, phenacyl linkers, alkoxybenzoin linkers, chromium arene complex linkers, NpSSMpact linkers and pivaloylglycol linkers, as described in Guillier et al. (Chem. Rev. 2000 1000:2091-2157). For example, a 1-(2-nitrophenyl)ethyl-based photocleavable linker (Ambergen) can be efficiently cleaved using near-UV light, e.g., achieving >90% yield in 5-10 minutes using a 365 nm peak lamp at 1-5 mW/cm2. In some embodiments, the modifiable group is a photocleavable group such as a nitro-aryl group, e.g., a nitro-indole group or a nitro-benzyl group, including but not limited to: 2-nitroveratryloxycarbonyl, a-carboxy-2-nitrobenzyl, 1-(2-nitrophenyl)ethyl, 1-(4,5-dimethoxy-2-nitrophenyl)ethyl and 5-carboxymethoxy-2-nitrobenzyl. Nitro-indole groups of interest include, e.g., a 3-nitro-indole, a 4-nitro indole, a 5-nitro indole, a 6-nitro-indole or a 7-nitro-indole group, where the indole ring may be further substituted at any suitable position, e.g., with a methyl group or a halo group (e.g., a bromo or chloro), e.g., at the 3-, 5- or 7-position. In certain embodiments, the nitro-aryl group is a 7-nitro indolyl group. In certain instances, the 7-nitro indolyl group is further substituted with a substituent that increases the photoactivity of the group, e.g., substituted with a bromo at the 5-position. Any convenient photochemistry of nitroaryl groups may be adapted for use. In certain embodiments, the linker includes a photocleavable group, such as a nitro-benzyl protecting group or a nitro-indolyl group.

In a given plurality of barcoded beads, the one or more domains of the barcoded reverse primers attached to different beads of the plurality may be identical or common among the barcoded beads. For example, the barcoded reverse primers of a given plurality may include the same or common anchor domain, which domain may be employed for binding to universal PCR primers and for follow-up amplification of barcoded extended DNA fragments. Other domains that may be common among the barcoded oligonucleotides include template-binding domains, e.g., in embodiments where the reverse primers include a single consensus sequence, such as oligo dT, linker domains, sample domains, etc.

For the purpose of capturing (binding) single cells of interest, in some instances the barcoded beads also include, in addition to the reverse primers, a moiety capable of binding to a target cell of interest from cell sample, i.e., a cellular binding moiety. When present, the cellular binding moiety may vary, and may be a moiety capable of specific binding to cell or a structural component thereof. Examples of cellular binding moieties of interest include, but are not limited to: lipids, e.g., which bind to the lipid layer of cell membrane, aptamers, and proteinaceous specific binding members, e.g., antibodies or specific binding fragments thereof, which bind to a specific antigen on cell surface or nucleus surface. The cellular binding moiety may be bound directly to bead surface or bind (covalently or non-covalently) indirectly to oligonucleotides attached to beads, e.g., such that is bound to the bead surface of an oligonucleotide linker. In some instances, specific antibodies are coupled to an oligonucleotide and incubated with the beads carrying a complementary docking oligonucleotide, creating beads capable of directed binding to the surface of cells expressing the antigen(s) recognized by the antibodies docked to the beads via the coupled oligonucleotide sequence. Specific cell binding moiety domains of interest include, but are not limited to, antibody binding agents, proteins, peptides, haptens, nucleic acids, aptamers, lipids, etc. The term “antibody binding agent” as used herein includes polyclonal or monoclonal antibodies or fragments that are sufficient to bind to an analyte of interest. The antibody fragments can be, for example, monomeric Fab fragments, monomeric Fab′ fragments, or dimeric F(ab)′2 fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies. The marker of the cell of interest may be any convenient marker, such as a cell surface protein or structure having an epitope to which the specific binding domain may specifically bind. In such instances, the bead linked sample barcoded reverse primers may include one or more additional domains of interest, such as bead identifying domains (bead barcodes), antibody identifying domains (antibody barcodes), etc.

In one embodiment, the antibodies used can be one or both of a pair of antibodies selected for universal binding of a variety of human cells (e.g., anti-beta-2-microglobulin, anti-CD298). In other embodiments, antibodies specific for cell populations of interest can be used to limit binding of beads to specific cells, (e.g., anti-CD14 for blood monocytes). In some embodiments, several bead sets wherein each set includes an antibody for a specific cell type may be combined and used in the disclosed assay together. For these multiplex cell typing applications, the oligonucleotides attached to antibody (or barcoded oligonucleotides attached to beads) could comprise the antibody-specific barcode domain which will allow to incorporate these antibody-specific barcode in barcoded DNA extension products.

In other embodiments, the cellular binding moiety capable of mediating binding to specific types or cells in general can be used to prepare beads for specific binding of cells for subsequent genetic analysis. These cell binding moieties include, but are not limited to: lipids (e.g., as described in McGinnis et al., “MULTI-seq: sample multiplexing for single-cell RNA sequencing using lipid-tagged indices,” Nat Methods. (2019); 16(7):619-26); lectins (etc., as described in Christiansen et al., “Identification of the major lectin-binding surface proteins of human neutrophils and alveolar macrophages,” Blood. (1988)71:1624-32); avidin (e.g., as described in Crupi et al., “Cell surface biotinylation of receptor tyrosine kinases to investigate intracellular trafficking,” Methods Mol Biol. (2015)1233:91-102); aptamers (e.g., as described in Zumrut et al., “Ligand-Guided Selection of Target-Specific Aptamers: A Screening Technology for Identifying Specific Aptamers Against Cell-Surface Proteins,” Nucl Acid Ther. (2016) 26:190-6); or other ligands for cell surface receptors or structures.

In one embodiment, barcoded beads with attached cell-specific binding moieties (e.g. antibodies) are incubated with suspensions containing the cells of interest, which could comprise all the cells within the suspension or a subset thereof. After binding, any resultant cell/barcoded bead complexes (e.g., made up of a single cell and single bead such as described above) may be isolated by flow cytometric sorting or used directly in the disclosed assay. Flow sorting allows one to employ various parameters to sort only specific cell population(s), e.g., antigen-specific cell fraction (e.g., CD45 cells) or sorting based on exclusion of fluorescent dyes to only sort live cell-bead complexes and exclude dead-cell-bead complexes.

As summarized above, methods of include combining a cellular sample with a plurality of distinct barcoded beads comprising barcoded reverse primers under conditions sufficient to produce a liquid composition comprising a plurality of separated cell/barcoded bead complexes, e.g., as described above. In some instances, barcoded beads, optional including cellular binding moiety (e.g. antibodies), such as described above, are incubated with a cellular made up of cells, where all of the cells of the cellular sample may be interest, or only a portion of the cells of the cellular sample may be of interest. Cell/barcoded bead complexes may be prepared so as to distribute them at distances from each other that minimize the diffusion of molecules from one cell/barcoded bead complex to another when present under hybridization conditions, e.g., as described in greater detail below.

In some instances, the cell/barcoded bead complexes are randomly distributed in a liquid composition, (e.g., an aqueous media). In such instances, the cell/barcoded bead complexes may be suspended in the liquid composition, separated from each other by distances that limit oligonucleotide diffusion between complexes, such as described above. In such embodiments, the number of complexes per ml of liquid composition may vary, ranging in some instances from 500 to 50,000, such as 1,000 to 20,000. In some instances, the liquid composition includes a stimulus-responsive polymer, e.g., as described in greater detail below.

In some instances, the cellular sample is combined with the plurality of distinct barcoded beads in a manner such that the resultant cell/barcoded bead complexes are separated from each other on a surface of a solid support. For example, a population of single cells may be attached to a solid surface, e.g., a surface commonly used in cell culture experiments (plastic, glass, etc.), where the distance between any cells on the surface exceeds the expected diffusion distance of barcoded reverse primers or template DNA or RNA released from cells. The solid surface could be non-modified or chemically modified, e.g., to create a pattern of hydrophilic areas separated by hydrophobic spacers separating hydrophilic areas from each other. A surface could be planar or with some elevation/depression which allows one to separate the cells from each other at distance exceeding diffusion distance of barcoded reverse primers. Furthermore, the surface may be modified with agents which have affinity for cells, e.g., gelatin, fibronectin, antibodies to cell-surface antigens, etc. After attachment, the attached cells may be incubated with barcoded beads, which may include a cellular binding moiety, to produce cell/barcoded bead complexes attached to the surface. Cell media with unbound beads may then be removed and replaced with a second media, which may include a stimulus-responsive polymer, such as described below.

In some instances, embodiments of the methods may employ a stimulus-responsive polymer, where the stimulus response polymer allows for preparation of cell/barcoded bead complexes in a liquid composition under mixing conditions suitable to achieve separation of cell/barcoded bead complexes by a desired distance, e.g., as described above. Following preparation of the separated cell/barcoded bead complexes, a suitable stimulus may be applied to the polymer to convert the polymer to a solid state, which limits the diffusion of barcoded reverse primers between cell/barcoded bead complexes. By application of the stimulus (e.g., temperature change) the mixture of polymer and bead-cell complexes undergoes a rapid phase change/transition that increases viscosity and immobilizes the cell/barcoded bead complexes randomly distributed within the matrix.

In such embodiments, any convenient stimulus responsive polymer may be employed. The desirable characteristics of the stimulus-responsive polymers include compatibility with cells and aqueous buffers, a lack of toxicity for cells, and absence of any interaction of the polymer with cells that might perturb the state of the cell and alter its transcriptome as a result. In addition, the desirable characteristics of the stimulus-responsive polymers include permissiveness for a limited amount of diffusion of small molecules, for example to allow introduction and delivery to the cell/barcoded bead complexes of cell lysis reagents. However, the stimulus-responsive polymer matrix should slow diffusion sufficiently to inhibit or preclude diffusion of barcoded reverse primers and nucleic acids from one cell/barcoded bead complex to another.

In one embodiment, solutions of methylcellulose molecules provide stimulus-responsive polymers that easily mix with and distribute cell/barcoded bead complexes at room temperature and then rapidly change phase upon exposure to increased temperature (e.g., 60° C.) creating a semi-solid matrix (“gelification”). In another embodiment, solutions of stimulus-responsive polymers are applied to adherent cells bound to barcoded beads to provide permissiveness for a limited amount of diffusion, for example to allow introduction and delivery to the cell/barcoded bead complexes of cell lysis reagents. However, the stimulus-responsive polymer matrix will slow diffusion sufficiently to preclude diffusion of barcoded reverse primers from one cell/barcoded bead complex to another.

In other embodiments, solutions of poly(N-isopropylacrylamide (PIPA) or other well known in art compositions provide stimulus-responsive polymers that may also be used to create a semi-solid matrix (“gelification”) containing well-dispersed bead-cell pairs.

In some instances, the stimulus response polymers are reversible, such that upon removal of the applied stimulus, e.g., heat, they return to their initial, soluble state upon exposure of cell-bead complexes embedded in gel at room temperature.

### Production of Primed Template Nucleic Acids

As reviewed above, following production of a plurality of separated cell-barcoded bead complex and, when present, phase change of any stimulus responsive polymer, the methods then include producing primed template nucleic acids by hybridizing template binding domains of barcoded reverse primers to template nucleic acids of the cells of the cell/barcoded bead complexes to produce primed template nucleic acids. Depending on a given protocol, the template nucleic acids of the primed template nucleic acids may vary. Essentially any nucleic acid template may find use in the subject methods, including e.g., RNA template nucleic acid and DNA template nucleic acids. RNA template nucleic acids may vary and may include e.g., messenger RNA (mRNA) templates, and the like. In addition, various types of DNA templates may be employed, including but not limited to e.g., genomic DNA templates, mtDNA templates, synthetic DNA templates, etc.

According to certain embodiments, the template nucleic acids are template ribonucleic acids (template RNA). Template RNAs may be any type of natural or/and artificial RNA or their combination present in cell sample. Natural RNA (or sub-type thereof) including, but not limited to, a messenger RNA (mRNA), a microRNA (miRNA), a transacting small interfering RNA (ta-siRNA), a natural small interfering RNA (nat-siRNA), a small nucleolar RNA (snoRNA), a small nuclear RNA (snRNA), a long non-coding RNA (IncRNA), a non-coding RNA (ncRNA), a transfer-messenger RNA (tmRNA), a precursor messenger RNA (pre-mRNA), a small Cajal body-specific RNA (scaRNA), a piwi-interacting RNA (piRNA), a small temporal RNA (stRNA), a signal recognition RNA, a telomere RNA, or any combination of RNA types thereof or subtypes thereof. Examples of artificial RNA which is usually delivered by synthetic or expressed from effector genetic constructs in a cell sample include, but are not limited to, a short hairpin RNA (shRNA), an endonuclease-prepared siRNA (esiRNA), a micro RNA, a small interfering RNA (siRNA), a single guide RNA (sgRNA), ribozyme, RNA encoding natural and genetically modified peptides, aptamers, proteins, clonal barcodes, UMI, genetic construct specific barcode (e.g., barcoded transcriptional reporter construct), regulatory RNA which could affect biological processes in target cell (e.g., as described in U.S. Pat. Nos. 9,429,565 and 10,196,634 (the disclosures of which are herein incorporated by reference) etc.

According to certain embodiments, the template nucleic acids are template deoxyribonucleic acids (template DNA). A template DNA may be any type of natural or genetically engineered DNA of interest to a practitioner of the subject methods, including but not limited to genomic DNA or fragments thereof, complementary DNA (or “cDNA”, synthesized from any RNA or DNA of interest), recombinant DNA (e.g., plasmid DNA), or the like.

To provide for access of the barcoded reverse primers of the barcoded beads to nucleic acids in the cells, the cell/barcoded bead complexes, which may be entrapped in a polymeric matrix, may be subjected to cell lysis/denaturation conditions which initiate interaction between cellular nucleic acids, e.g., mRNAs, and barcoded reverse primers. Where desired, chemical agents may be employed to lyse cells within the semi-solid matrix to allow release of nucleic acids (e.g., RNA). In one embodiment, Qiagen TCL buffer is applied to the surface of the semi-solid matrix, where the buffer diffuses into the matrix to disrupt the cells within the cell/barcoded bead complexes present in the matrix so as to release the cellular RNA molecules for binding to the barcoded reverse primers provided by the barcoded bead of the complex. The cell lysis/hybridization step may be initiated by changing media surrounding cells with cell lysis solution using any convenient lysis composition, such as a cell lysis buffer solution containing denaturing agents (e.g., guanidium thiocyanate, urea, etc.), detergents (SDS, triton X100, NP40, etc.), hybridization accelerators (salt, polyethylene glycol, etc.), additives (EDTA, proteinase K, nuclease inhibitors, etc.) and the like. Any suitable lysis method may be employed. In some instances, a mild lysis procedure can advantageously be used to prevent the release of nuclear chromatin, thereby avoiding genomic contamination of a cDNA library, and to minimize degradation of mRNA. For example, heating cells at 60-70° C. for 2 minutes in the presence of Tween-20 is sufficient to lyse the cells while resulting in no detectable genomic contamination from nuclear chromatin. Alternatively, cells can be heated to 65° C. for 10 minutes in water (Esumi et al., Neurosci Res 60(4):439-51 (2008)); or 70° C. for 90 seconds in PCR buffer II (Applied Biosystems) supplemented with 0.5% NP-40 (Kurimoto et al., Nucleic Acids Res 34(5):e42 (2006)); or lysis can be achieved with a protease such as Proteinase K or by the use of chaotropic salts such as guanidine isothiocyanate (U.S. Patent application Publication No. 2007/0281313). Additional mild-lysis conditions, which do not destroy but permeabilize the cellular membrane, like treatment with methanol, detergents (Triton X-100, Tween-20, etc.), may be employed to initiate hybridization between barcoded reverse primers and cellular RNAs.

Treatment of cell/barcoded bead complexes under lysis conditions causes release of or otherwise makes accessible the nucleic acids (e.g., mRNA) from the single cells of the cell/barcoded bead complexes. This allows binding of RNA molecules to the barcoded reverse primers provided by barcoded bead of the complex. In one embodiment, the barcoded reverse primers are released from the barcoded bead by cleavage, such as by exposure to light (e.g., UV light) to activate cleavage of a photosensitive linker, causing release of the barcoded reverse primers to allow greater interaction and hybridization with their complementary partners within the pool of cellular RNA now available for binding (e.g., after cell lysis by physical/chemical means). In another embodiment, the barcoded reverse primers are not detached from the beads and hybridize to RNA molecules on the surface of the barcoded beads. In some embodiments, the hybridization step includes treatment of DNA and in some instances of RNA to make it more accessible to hybridization with barcoded reverse primers. These treatments may include any of a number of protocols, including fragmentation of DNA (e.g., ultrasound), treatment with enzymes (e.g., Proteinase K), heat denaturation (e.g., 95 C for 1 min), and the like. In some instances, the lysis and hybridization step is one step as lysis buffer composition may include the components which are necessary for hybridization step. The hybridization conditions (temperature, buffer compositions, time) may be optimized in order to provide high efficiency and specific interaction between template-binding domains (e.g., oligo dT or gene-specific domains) and nucleic acid.

As a result of hybridization, a plurality of primed template nucleic acids are produced for each cell/barcoded bead complex, which plurality of primed template nucleic acids is made up of hybridized nucleic acids comprising a template nucleic acid, e.g., mRNA or genomic DNA fragment, hybridized to a barcoded reverse primer. The number of different primed template nucleic acids which differ from each other at least in terms of the template nucleic acid sequence may vary, where in some instances the number of distinct primed template nucleic acids in the plurality of primed template nucleic acids ranges from 1 to 200,000, 10 to 25,000, such as 100 to 20,000 and including 1,000 to 10,000, 10,000 to 20,000, 15,000 to 20,000 and 15,000 to 19,000. In some instances, the different primed template nucleic acids share common barcoded reverse primers, e.g., where the reverse primers include consensus template binding domains, e.g., oligo dT domains. In other instances, the different primed template nucleic acids have different barcoded reverse primers hybridized thereto, e.g., where the barcoded reverse primers are gene specific barcoded reverse primers.

Following production of the primed template nucleic acids from the disparate cell/barcoded bead complexes, the primed template nucleic acids from one or more different cell/barcode bead complexes may be combined or pooled for further processing. In such pooled compositions, each plurality of primed template nucleic acids derived from single cell/barcoded bead complex of the pooled composition will have a distinct barcode domain, such that the barcode domain of a first plurality of primed template nucleic acids of the composition will have a sequence that differs from every other barcode domain of every other plurality of primed template nucleic acids in the pooled composition. In a given pooled composition, each barcode domain has a sequence that is significantly different from that of any other barcode domain in the pooled composition, with a difference of at least 1 nucleotide, such as 2 nucleotides and including 3 or more nucleotide differences in the whole set of barcodes employed in the assay. In this way each plurality of the pooled composition will have a distinct identifying barcode domain. The number of different barcode domains in such pooled compositions is the same as the number of different pluralities in the pooled composition, where the number represents the number of different samples that is employed to make the pooled composition. The number of different barcodes present in a given pooled composition depends on number of samples being analyzed in a given assay. In some instances, the number ranges from 10 to 1,000,000, such as 100 to 100,000, and including 1,000 to 10,000. For example, currently for analysis of single-cell samples, the number of barcodes may be 10,000 or more, but for analysis of clinical samples the number of barcodes may not exceed 1,000.

Where desired, hybridization complexes of template and primer, i.e., primed template nucleic acids, may be purified, e.g., via separation from excess of non-bound primers, e.g., by nuclease treatment or/and binding to solid support, e.g., such as beads, e.g., as described below. In this way, excess of primers, such as oligo dT primers and/or gene-specific primers, may be removed in order to achieve a high specificity of primer extension reaction from the target template sequences. In some embodiments, prior to subject to primer extension reaction conditions, e.g., as described in greater detail below, the plurality of primed template nucleic acids are combined together and purified from other constituents that may be present in the reaction mixture, such as non-bound barcoded reverse primers, non-hybridized nucleic acids, proteins, reverse transcriptase inhibitors, and the like. In those instances where a stimulus-responsive polymer has been employed, the polymer matrix with entrapped cell/barcoded bead compositions is converted to liquid form by removing the stimulus condition (e.g., by cooling down the composition to room temperature for temperature-responsive polymers, like methylcellulose, such as described above). The purification of primed template nucleic acids may be achieved using any convenient protocol, e.g., by binding to a matrix, via fractionation based on size, charge, solubility, precipitation, etc. In one embodiment, the primed template nucleic acids are purified using oligo dT-magnetic beads, followed by centrifugation or magnet binding steps and washing steps. In another embodiment, the primed template nucleic acids are separated from other components in the reaction mixture by contacting the mixture with a matrix (e.g., AMPure XP magnetic beads, glass particles (Qiagen), anion exchange resin(Qiagen), etc.) which specifically binds RNA or/and DNA molecules under optimized conditions but does not bind barcoded reverse primers. Some other protocols that may be employed include centrifugation, chromatography, precipitation, phase separation, etc. As a result of purification step, the plurality of prime template nucleic acids derived from different cells are purified from other components of the reaction mixture, e.g., non-bound oligonucleotides and other cellular components. The resultant purified primed template nucleic acids may be combined or pooled together, e.g., in a small volume of buffer, for subsequent primer extension to produce barcoded nucleic acids, e.g., as described above.

### Production of Barcoded Nucleic Acids

In methods of the invention, following production of the plurality of primed template nucleic acids, e.g., as described above, the primed template nucleic acids are subjected to primer extension reaction conditions sufficient to produce barcoded nucleic acids. The barcoded nucleic acids produced in this step include at least first strand cDNA flanked at one end, i.e., the 5′ end, with, among other optional domains, a reverse primer domain, a barcode domain and anchor domain, which domains have been provided to the barcoded nucleic acid by a barcoded reverse primer of a barcoded bead.

As reviewed above, in producing barcoded nucleic acids, primed template nucleic acids, e.g., as described above, are subjected to primer extension reaction conditions sufficient to produce the barcoded nucleic acids. By “primer extension reaction conditions” is meant reaction conditions that permit polymerase-mediated extension of a 3′ end of a nucleic acid strand, e.g., a barcoded reverse primer, hybridized to a template nucleic acid. Achieving suitable reaction conditions may include selecting reaction mixture components, concentrations thereof, and a reaction temperature to create an environment in which the polymerase is active and the relevant nucleic acids in the reaction interact (e.g., hybridize) with one another in the desired manner.

In producing the primer extension reaction mixture, the primed template nucleic acids may be combined with a number of additional reagents (e.g., to increase specificity, uniformity, yield, etc. of extension products), which may vary as desired. A variety of polymerases may be employed when practicing the subject methods. Reference to a particular polymerase, such as those exemplified below, will be understood to include functional variants thereof unless indicated otherwise. Examples of useful polymerases include DNA polymerases, e.g., where the template nucleic acid is DNA. In some instances, DNA polymerases of interest include, but are not limited to: thermostable DNA polymerases, such as may be obtained from a variety of bacterial species and genetically modified to improve their performance, including Thermus aquaticus (Taq), Thermus thermophilus (Tth), Thermus filiformis, Thermus flavus, Thermococcus literalis, and Pyrococcus furiosus (Pfu) or modified and mutated versions of these DNA polymerases (e.g. Phusion DNA polymerase, Q5 DNA polymerase, etc.). Alternatively, where the target template nucleic acid composition is made up of RNA, the polymerase may be a reverse transcriptase (RT), where examples of reverse transcriptases include natural and genetically modified versions of Moloney Murine Leukemia Virus reverse transcriptase (MMLV RT), e.g., SuperScript II, SuperScript III, Maxima reverse transcriptase (Thermo-Fsher), SMARTScribe™ reverse transcriptase (Takara), AMV reverse transcriptase, Bombyx mori reverse transcriptase (e.g., Bombyx mori R2 non-LTR element reverse transcriptase), etc. In one embodiment, the enzymes with DNA polymerase activity are designed for hot-start primer extension reaction, e.g., used as a complex with specific antibody or chemical compound which blocks enzymatic activity at low temperature but fully releases the activity at reaction conditions. For example, in some instances a hot-start reverse transcriptase composition, e.g. complex between MMLV RT and Therma-Stop RT reagent (Thermagenix) or complex between MMLV RT and antibody is employed.

Primer extension reaction mixtures also include dNTPs. In certain aspects, each of the four naturally-occurring dNTPs (dATP, dGTP, dCTP and dTTP) are added to the reaction mixture. For example, dATP, dGTP, dCTP and dTTP may be added to the reaction mixture such that the final concentration of each dNTP is from 0.05 to 10 mM, such as from 0.1 to 2 mM, including 0.2 to 1 mM. According to one embodiment, at least one type of nucleotide added to the reaction mixture is a non-naturally occurring nucleotide, e.g., a modified nucleotide having a binding or other moiety (e.g., a fluorescent moiety) attached thereto, a nucleotide analog, or any other type of non-naturally occurring nucleotide that finds use in the subject methods or a downstream application of interest.

In addition to the primed template nucleic acids, the polymerase, and dNTPs, the reaction mixture may include buffer components that establish an appropriate pH, salt concentration (e.g., KCl concentration), metal cofactor concentration (e.g., Mg²⁺ or Mn²⁺ concentration), and the like, for the extension reaction and template switching to occur. Other components may be included, such as one or more nuclease inhibitors (e.g., an RNase inhibitor and/or a DNase inhibitor), one or more additives for facilitating amplification/replication of GC rich sequences (e.g., GC-Mer™ reagent (Takara Bio USA (Mountain View, Calif.)), betaine, single-stranded binding proteins (e.g., T4 Gene 32, cold shock protein A (CspA), recA protein, and/or the like) DMSO, ethylene glycol, 1,2-propanediol, or combinations thereof), one or more molecular crowding agents (e.g., polyethylene glycol, or the like), one or more enzyme-stabilizing components (e.g., DTT present at a final concentration ranging from 1 to 10 mM (e.g., 5 mM)), and/or any other reaction mixture components useful for facilitating polymerase-mediated extension reactions.

The primer extension reaction mixture can have a pH suitable for the primer extension reaction. In certain embodiments, the pH of the reaction mixture ranges from 5 to 9, such as from 7 to 9, including from 8 to 9, e.g., 8 to 8.5. In some instances, the reaction mixture includes a pH adjusting agent. pH adjusting agents of interest include, but are not limited to, sodium hydroxide, hydrochloric acid, phosphoric acid buffer solution, citric acid buffer solution, and the like. For example, the pH of the reaction mixture can be adjusted to the desired range by adding an appropriate amount of the pH adjusting agent.

The temperature range suitable for production of the product nucleic acid may vary according to factors such as the thermal stability of particular polymerase employed, the melting temperatures of any primers employed, etc. According to one embodiment, the primer extension reaction conditions include bringing the reaction mixture to a temperature ranging from 4 to 72° C., such as from 16 to 70° C., e.g., 37 to 65° C., such 30 as 55° to 65° C. The temperature of the reaction mixture may be maintained for a sufficient period of time for polymerase mediated, template directed primer extension to occur. While the period of time may vary, in some instances the period of time ranges from 5 to 60 minutes, such as 15 to 45 minutes, e.g., 30 minutes.

In some embodiments, the primer reaction extension conditions using RNA template may incorporate a template switching oligonucleotide, e.g., with sample-specific barcode domain and anchor domain. Template switch is described in U.S. Pat. Nos. 5,962,271 and 5,962,272, as well as Published PCT application Publication No. WO2015/027135; the disclosures of which are herein incorporated by reference. Where desired, the template switch oligonucleotide may be employed to introduce one or more domains at the 3′ end of the cDNA, such as but not limited to, an anchor domain, an adaptor domain or portion thereof, sample barcode domain, etc., e.g., as described in United States Published Patent Application Nos. 20150111789 and 20150203906, the disclosures of which are herein incorporated by reference. Template switch oligonucleotides may be employed in protocols where forward primers are not used, as desired.

The resultant barcoded nucleic acid may, where desired, be contacted with a one or more forward primers, e.g., to introduce one or more desired domains to the end of the barcoded nucleic acid, where such domains may vary. In some instances, one or more forward primers is employed in an additional primer extension reaction to introduce a second anchor domain at the end of the barcoded nucleic acid that is opposite the end that includes the first anchor domain, e.g., to produce “sample-barcoded anchor-domain-flanked deoxyribonucleic acid (DNA) fragments”, by which is meant a DNA which is derived from genomic DNA or RNA templates and includes an anchor domain on each side of a gene-specific domain. In these instances, the forward primer(s) may vary. In some instances, a single forward primer is employed, where the primer includes a template binding domain that binds all or a desired conservative sequence/portion of the primer extension products from the first strand synthesis, e.g., where the template binding domain binds to a common sequence provided by a template switch oligonucleotide employed in first strand synthesis. Alternatively, a plurality of different forward primers may be employed, such as a collection for forward gene specific primers, e.g., that include a common anchor domain 5′ of a unique gene specific domain. While the number of distinct primers in a given set may vary, as desired, in some instances the number of primers in a given set is 10 or more, such as 20 or more, 30 or more, 40 or more, 50 or more, 60 or more, 70 or more, 80 or more, 90 or more, 100 or more, 125 or more, 250 or more, 500 or more, including 1000 or more, 200 or more, 5000 or more, 8000 or more, 10,000 or more 15,000 or more, 18,000 or more and 20,000 or more. In some instances, the number of gene specific primers that is present in the set is 25,000 or less, such as 20,000 or less. As such, in some embodiments the number of gene specifics in the set that is employed in the methods ranges from 10 to 25,000, such as 50 to 20,000, including 1,000 to 10,000, e.g., 2,500 to 8,500, and 10,000 to 20,000, e.g., 15,000 to 19,000. Gene specific reverse primers include gene specific domains, where these gene specific domains may be experimentally validated as suitable for use in a multiplex amplification assay. By “experimentally validated as suitable for use in a multiplex amplification assay” is meant that primers for each target gene in a given set has been experimentally tested in a multiplex amplification assay, such as described in United States Published Patent Application Nos. 20160376664 and 20180245164, the disclosures of which are herein incorporated by reference. To control efficiency and specificity of primer hybridization and the subject extension step, the length of the gene specific domain of the gene specific primer may vary. In some instances, the length ranges from 10 to 120 nt, such as 15 to 75 nt, e.g., 16 to 50 nt, such as 18 to 40 nt, including 20 to 30 nt or 25 to 40 nt. The gene specific domain primer may vary length. In some instances, the length of the gene specific domain in the forward primers ranges from 16 to 40 nt, such as 18 to 30 nt. As the gene specific primers are barcoded and may include additional domains, e.g., anchor domains, etc., in some embodiments the primers in length from 20 to 150 nt, such as 25 to 100 nt, including 27 to 75 nt, such as from 30 to 60 nt, including from 30 to 50 nt. Where desired, the gene specific primers may be GCA- and/or GCT-rich. By GCA- and/or GCT-rich is meant that the gene-specific primer domain has a substantial portion of G, C, A- and/or G, C, T nucleotides. While the number of such nucleotides in a gene specific primer domain may vary, in some instance the number of such sequences ranges from 75% to 100%, such as 85% to 100%. As the gene specific primer domains of such embodiments are GCA- and/or GCT-rich, the GC content of the gene specific primer domains is also high. While the GC content may vary, in some instances the GC content ranges from 40 to 90%, such as 45 to 85%, including 50 to 85%, e.g., 50 to 80%. Depending on the specific application for which the set is configured, the set of gene specific primers may be configured to target a wide range of mammalian genes, genetically modified genes or artificial or recombinant sequences (e.g. barcodes, genes, effector constructs) introduced in the cells, and pathogenic genes from a wide range of pathogenic organisms, such as viruses, bacteria, fungi, etc. which could be present in the human or mammalian bodies. Of interest in certain applications are human, mammalian species commonly used as a model organisms to study human diseases, such as mouse, rat, or monkey, and pathogenic organisms associated with human diseases. To be analyzed in accordance with embodiments of the invention, the targeted genes may be present in the mammalian cells or biological fluids, e.g. exosomes, circulating tumor DNA, etc. In some embodiments, the targeted genes are may be protein coding, or may express non-coding RNAs, micro RNAs, mitochondrial RNAs, regulatory RNAs, etc. In some instances, the set of genes selected is genome-wide, such that it covers all genes present in the genome of an organism. In other embodiments, the genes are selected from the genes that could be transcribed or expressed in the organism and present in the biological samples in the form of RNA. The genome-wide set of genes specific for human, model and pathogenic organisms is of special interest in some instances and may be used to develop a set of genome-wide targeted RNA expression assays based on the disclosed multiplex PCR assay. Genome-wide sets of primers may vary in number, and in some instances are configured to assay 18,000 or more, such as 20,000 or more and 25,000 or more, such as 30,000 or more genes. Additional sets of PCR primers may be configured based on a genome-wide set of genes from a wide range of viral, bacterial and eukaryotic pathogenic organisms. In another embodiment, the gene specific primers may be configured to produce primer extension products from a subset of specific genes selected from the genome-wide set of genes. Examples of sets of gene-specific primers and their use in single cell genetic analysis applications is disclosed in U.S. patent application Ser. Nos. 15/133,184 and 16/543,211, the disclosures of which sets of gene specific primers are incorporated herein by reference.

In the sample-barcoded anchor-domain-flanked deoxyribonucleic acid (DNA) fragments produced by embodiments of the invention, the gene-specific domain is one or several DNA fragments derived from one gene encoded by genomic DNA or RNA template. Where gene specific primers are employed, e.g., as described above, the gene-specific domain may be specific sequence flanked from one or both sides with specific sequences of forward and reverse gene-specific primers. In one embodiment, the gene-specific domain is flanked from both sides by gene-specific primer sequences. In another embodiment, the gene-specific domain may correspond to the 3′-end sequence of an mRNA and be flanked from 3′-end by oligo dT sequences and from the other end by gene-specific primer or by anchor domain sequences which is non-specifically attached to an arbitrary gene sequence upstream of 3′-mRNA end (e.g., through ligation of anchor adaptor using transposase). A non-specific anchor domain may be also attached to the 5′-end of mRNA using e.g., template switch technology to provide a gene-specific domain flanked by one anchor domain at the 5′-end of mRNA molecule and gene-specific primer sequence or another non-specific to the sequence anchor domain.

As the gene-specific domain is flanked by anchor domains in these embodiments, the DNA fragments prepared by methods of the invention include a first anchor domain located at a first end of the DNA fragment and a second anchor domain located at a second end of the DNA. By gene-specific domain is meant a region of the dsDNA fragment the includes a sequence found in template nucleic acid, such as a template mRNA or DNA. While the length of the gene domain may vary, in some instances the gene domain ranges in length from 50 to 500 nt, such as 60 to 300 nt.

In addition to the gene-specific domains, as described above, the DNA fragments have anchor domains on either side of the gene domain. Anchor domains are domains that are employed in nucleic acid amplification, such as polymerase chain reaction (PCR), steps of the methods, where they serve as primer binding sites for the primers employed in such amplification steps, e.g., as described above. As summarized above, the DNA fragments are also “sample-barcoded”, by which is meant that they include a barcode domain that denotes, i.e., indicates or provides, information about (such that it may be used to determine), the specific sample, e.g., cell, from which the fragment has been produced, where the barcode domains are provided by the barcoded beads of the cell/barcoded bead complexes, e.g., as described above. As reviewed above, barcode domains include unique, specific sequences. While the length of a given barcode domain may vary, in some instances the length ranges from 6 to 30 nt, such as 8 to 20 nt, and including 12 to 18 nt. In addition to the gene-specific, barcode and anchor domains, the fragments produced by methods of the invention may further include additional domains, such as but not limited to a UMI domain, a linker domain, an adaptor domain, etc.

Embodiments of the methods may be characterized as methods of preparing a plurality of sample-barcoded anchor-domain-flanked DNA fragments from a template nucleic acid sample, e.g., a template ribonucleic acid (template RNA) sample. More specifically the methods may be characterized as multiplex methods of preparing a plurality of sample-barcoded anchor-domain-flanked gene specific deoxyribonucleic acid DNA fragments from a template nucleic acid, e.g., RNA, sample, such that each DNA fragment of the plurality is produced at the same time from the RNA or DNA sample, e.g., each DNA fragment is produced simultaneously from the source RNA or DNA sample. The number of distinct DNA fragments prepared in a given method may vary, where in some instances the number in the plurality ranges from 1 to 200,000, 10 to 25,000, such as 100 to 20,000 and including 1,000 to 10,000, 10,000 to 20,000, 15,000 to 20,000 and 15,000 to 19,000.

Among the DNA fragments of the plurality that are produced from a single sample by methods of the invention, a given DNA fragment is considered to be distinct from another DNA fragment if the gene-specific domains of the two fragments differ from each other by sequence. In some embodiments, the difference between two DNA fragments could be as small as one nucleotide, e.g., gene specific fragment with single nucleotide polyphormism (SNP) region. While the gene-specific domains of the DNA fragments in a given plurality may all differ from each other, e.g., because they include coding sequences of different genes, the DNA fragments will also include common domains, i.e., domains that are identical to each other (i.e., domains having sequences that do not differ from each other), where these domains are the flanking anchor domains, the barcode domains, etc. When employed, the DNA fragments may further differ with respect to additional domains, such as distinct UMI domains, such that the UMI domains of the DNA fragments have different sequences, i.e., they are not common or identical.

As indicated above, during a given protocol a plurality of DNA fragments produced from one sample may be combined, i.e., pooled, with one or more additional pluralities produced from one or more additional samples, e.g., plurality of single cells or nucleus derived from single cells. In such pooled compositions, each plurality of the pooled composition will have a distinct barcode domain, such that the barcode domain of a first plurality of the composition will have a sequence that differs from every other barcode domain of every other plurality in the pooled composition. In a given pooled composition, each barcode domain has a sequence that is significantly different from that of any other barcode domain in the pooled composition, with a difference of at least 1 nucleotide, such as 2 nucleotides and including 3 or more nucleotide differences in the whole set of barcodes employed in the assay. In this way each plurality of the pooled composition will have a distinct identifying barcode domain. The number of different barcode domains in such pooled compositions is the same as the number of different pluralities in the pooled composition, where the number represents the number of different samples that is employed to make the pooled composition. The number of different barcodes present in a given pooled composition depends on number of samples being analyzed in a given assay. In some instances, the number ranges from 10 to 1,000,000, such as 100 to 100,000, and including 1,000 to10,000. For example, currently for analysis of single-cell samples, the number of barcodes may be 10,000 or more, but for analysis of clinical samples the number of barcodes may not exceed 1,000.

### Amplification

In some instances, barcoded nucleic acids are amplified, where amplicons are produced from the barcoded nucleic acids produced by the primer extension step, e.g., as described above. The term “amplicon” is employed in its conventional sense to refer to a piece of DNA that is the product of artificial amplification or replication events, e.g., as produced using various methods including polymerase chain reactions (PCR), ligase chain reactions (LCR), rolling circle amplification (RCA), etc. Where primer extension products are amplified, the primer extension products, e.g., as described above, may include additional domains that are employed in subsequent amplification steps to produce a desired amplicon composition. For example, flanking anchor domains are provided in the primer extension products, where the flanking anchor domains include universal priming sites which may be employed in PCR amplification.

As such, embodiments of the methods may include combining a primer extension product composition of barcoded nucleic acids with universal forward and reverse primers under amplification conditions sufficient to produce a desired product barcoded amplicon composition. The forward and reverse universal primers may be configured to bind to the common forward and reverse anchor domains and thereby nucleic acids present in the primer extension product compositions. The universal forward and reverse primers may vary in length, ranging in some instances from 10 to 75 nt, such as 18 to 60 nt.

In some instances, the universal forward and reverse primers include one or more additional domains, such as but not limited to: an indexing domain, a clustering domain, a Next Generation Sequencing (NGS) adaptor domain (i.e., high-throughput sequencing (HTS) adaptor domain), etc. Alternatively, these domains may be introduced during one or more subsequent steps, such as one or more subsequent amplification reactions, e.g., as described in greater detail below. The amplification reaction mixture will include, in addition to the primer extension product composition and universal forward and reverse primers, other reagents, as desired, such polymerase, dNTPs, buffering agents, etc., e.g., as described above.

Amplification conditions may vary. In some instances, the reaction mixture is subjected to polymerase chain reaction (PCR) conditions. PCR conditions include a plurality of reaction cycles, where each reaction cycle includes: (1) a denaturation step, (2) an annealing step, and (3) a polymerization step. The number of reaction cycles will vary depending on the application being performed, and may be 1 or more, including 2 or more, 3 or more, four or more, and in some instances may be 15 or more, such as 20 or more and including 30 or more, where the number of different cycles will typically range from about 12 to 24. The denaturation step includes heating the reaction mixture to an elevated temperature and maintaining the mixture at the elevated temperature for a period of time sufficient for any double stranded or hybridized nucleic acid present in the reaction mixture to dissociate. For denaturation, the temperature of the reaction mixture may be raised to, and maintained at, a temperature ranging from 85 to 100° C., such as from 90 to 98° C. and including 94 to 98° C. for a period of time ranging from 3 to 120 sec, such as 5 to 30 sec. Following denaturation, the reaction mixture will be subjected to conditions sufficient for primer annealing to template DNA present in the mixture. The temperature to which the reaction mixture is lowered to achieve these conditions may be chosen to provide optimal efficiency and specificity, and in some instances ranges from about 50 to 75° C., such as 60 to 74° C. and including 68 to 72° C. Annealing conditions may be maintained for a sufficient period of time, e.g., ranging from 10 sec to 30 min, such as from 10 sec to 5 min. Following annealing of primer to template DNA or during annealing of primer to template DNA, the reaction mixture may be subjected to conditions sufficient to provide for polymerization of nucleotides to the primer ends in manner such that the primer is extended in a 5′ to 3′ direction using the DNA to which it is hybridized as a template, i.e. conditions sufficient for enzymatic production of primer extension product. To achieve polymerization conditions, the temperature of the reaction mixture may be raised to or maintained at a temperature ranging from 65 to 75, such as from about 68 to 72° C. and maintained for a period of time ranging from 15 sec to 20 min, such as from 20 sec to 5 min. In some embodiments, the annealing stage could be avoided, and protocol could include only denaturation and polymerization steps as described above. The above cycles of denaturation, annealing and polymerization may be performed using an automated device, typically known as a thermal cycler. Thermal cyclers that may be employed are described in U.S. Pat. Nos. 5,612,473; 5,602,756; 5,538,871; and 5,475,610, the disclosures of which are herein incorporated by reference.

The product amplicon composition of this first amplification reaction will include amplicons corresponding to the gene specific domains that are present in the initial target nucleic acid composition and are bounded by primer pairs present in the employed set of gene specific primers and barcode sequence from one side of the amplicon. In some instances, the number of distinct amplicons of differing sequence in this initial amplicon composition ranges from 10 to 19,000, 10 to 15,000, 10 to 10,000, and 10 to 8,000, such as 25 to 18,500, 25 to 12,000, 25 to 8,000, and 25 to 7,500, including 50 to 15,000, 50 to 10,000 and 50 to 5,000, where in some instances the number of distinct amplicons present in this initial amplicon composition is 25 or more, including 50 or more, such as 100 or more, 250 or more, 500 or more, 1,000 or more, 1,500 or more, 2,500 or more, 5,000 or more, 7,500 or more, 8,500 or more, 10,000 or more, 15,000 or more, 18,000 or more. A subject amplicon composition may include or exclude multiple different product amplicons corresponding to same gene as amplified by two or more different primer pairs directed to the gene. The multiple product amplicons making up the amplicon composition may vary in length, ranging in length in some instances from 50 to 1000, such as 60 to 500, including 70 to 250 nt. The sample barcoded initial product amplicon composition may be employed in a variety of different applications, including evaluation of the expression profile of the sample from which the template target nucleic acid was obtained. In such instances, the expression profile may be obtained from the amplicon composition using any convenient protocol, such as but not limited to differential gene expression analysis, array-based gene expression analysis, NGS sequencing, etc.

For example, the barcoded amplicon composition may be employed in hybridization assays in which a nucleic acid array that displays “probe” nucleic acids for each of the genes to be assayed/profiled in the profile to be generated is employed. In these assays, the amplicon composition is first prepared from the initial target nucleic acid sample being assayed as described above, where preparation may include labeling of the target nucleic acids with a label, e.g., a member of signal producing system.

Following amplicon production, e.g., as described above, the sample is contacted with the array under hybridization conditions, whereby complexes are formed between target nucleic acids that are complementary to probe sequences attached to the array surface. The presence of hybridized complexes is then detected, either qualitatively or quantitatively. The detection and quantification of different barcodes could be achieved in the follow-up hybridization steps with labeled targets complementary to barcode domains of the amplicons. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptome), may be both qualitative and quantitative.

Alternatively, non-array-based methods for quantifying the levels of one or more nucleic acids in a sample may be employed, including quantitative PCR, real-time quantitative PCR, and the like. (For general details concerning real-time PCR see Real-Time PCR: An Essential Guide, K. Edwards et al., eds., Horizon Bioscience, Norwich, U.K. (2004).

In some embodiments, the method further includes sequencing the multiple barcoded product amplicons, e.g., by using a Next Generation Sequencing (NGS) protocol. In such instances, if not already present, the methods may include modifying the initial amplicon composition to include one or more components employed in a given NGS protocol, e.g., sequencing platform adaptor constructs, indexing domains, clustering domains, etc.

By “sequencing platform adapter construct” is meant a nucleic acid construct that includes at least a portion of a nucleic acid domain (e.g., a sequencing platform adapter nucleic acid sequence) or complement thereof utilized by a sequencing platform of interest, such as a sequencing platform provided by Illumina® (e.g., the NovaSeq™, NexSeq™, HiSeq™, MiSeq™ and/or Genome Analyzer™ sequencing systems); Thermo Fisher (e.g., Ion Torrent™ (such as the Ion PGM™ and/or Ion Proton™ sequencing systems) and Life Technologies™ (such as a SOLiD sequencing system)); Pacific Biosciences (e.g., the PACBIO RS II sequencing system); Roche (e.g., the 454 GS FLX+ and/or GS Junior sequencing systems); Oxford Nanopore technologies (e.g., MinION™, GridION™′ PrometION™ sequencing systems) or any other sequencing platform of interest.

In certain aspects, the sequencing platform adapter construct includes a nucleic acid domain selected from: a domain (e.g., a “capture site” or “capture sequence”) that specifically binds to a surface-attached sequencing platform oligonucleotide (e.g., the P5/i5 or P7/i7 oligonucleotides attached to the surface of a flow cell in an Illumina® sequencing system); where the construct may include one or more additional domains, such as but not limited to: a sequencing primer binding domain or clustering domain (e.g., a domain to which the Read 1 or Read 2 primers of the Illumina® platform may bind); a indexing domain (e.g., a domain that uniquely identifies the sample source of the nucleic acid being sequenced to enable sample multiplexing by marking every molecule from a given sample with a specific index or “tag”); a barcode sequencing primer binding domain (a domain to which a primer used for sequencing a barcode binds); a unique molecular identification domain (e.g., a molecular index tag, such as a randomized tag of 4, 6, or other number of nucleotides) for uniquely marking molecules of interest to determine expression levels based on the number of instances a unique tag is sequenced; a complement of any such domains; or any combination thereof. In certain aspects, a barcode domain (e.g., sample index tag) and a molecular identification domain (e.g., a molecular index tag) may be included in the same nucleic acid.

The sequencing platform adapter constructs may include nucleic acid domains (e.g., “sequencing adapters”) of any length and sequence suitable for the sequencing platform of interest. In certain aspects, the nucleic acid domains are from 4 to 200 nucleotides in length. For example, the nucleic acid domains may be from 4 to 100 nucleotides in length, such as from 6 to 75, from 8 to 50, or from 10 to 40 nucleotides in length. According to certain embodiments, the sequencing platform adapter construct includes a nucleic acid domain that is from 2 to 8 nucleotides in length, such as from 9 to 15, from 16-22, from 23-29, or from 30-36 nucleotides in length. The nucleic acid domains may have a length and sequence that enables a polynucleotide (e.g., an oligonucleotide) employed by the sequencing platform of interest to specifically bind to the nucleic acid domain, e.g., for solid phase amplification and/or sequencing by synthesis of the cDNA insert flanked by the nucleic acid domains. Example nucleic acid domains include the P5 (5′-AATGATACGGCGACCACCGA-3′) (SEQ ID NO:03), P7 (5′-CAAGCAGAAGACGGCATACGAGAT-3′)(SEQ ID NO:04), Read 1 primer (5′-ACACTCTTTCCCTACACGACGCTCTTCCGATCT-3′) (SEQ ID NO:05) and Read 2 primer (5′-GTGACTGGAGTTCAGACGTGTGCTCTTCCGATCT-3′) (SEQ ID NO:06) domains employed on the Illumina®-based sequencing platforms. Other example nucleic acid domains include the A adapter (5′-CCATCTCATCCCTGCGTGTCTCCGACTCAG-3′)(SEQ ID NO:07) and P1 adapter (5′-CCTCTCTATGGGCAGTCGGTGAT-3′)(SEQ ID NO:08) domains employed on the Ion Torrent™-based sequencing platforms.

The nucleotide sequences of nucleic acid domains useful for sequencing on a sequencing platform of interest may vary and/or change over time. Adapter sequences are typically provided by the manufacturer of the sequencing platform (e.g., in technical documents provided with the sequencing system and/or available on the manufacturer's website). Based on such information, the sequence of the sequencing platform adapter construct of the template switch oligonucleotide (and optionally, a first strand synthesis primer, amplification primers, and/or the like) may be designed to include all or a portion of one or more nucleic acid domains in a configuration that enables sequencing the nucleic acid insert (corresponding to the template nucleic acid) on the platform of interest.

The sequencing adaptors may be added to the amplicons of the initial amplicon composition using any convenient protocol, where suitable protocols that may be employed include, but are not limited to: amplification protocols, ligation protocols, etc. In some instances, amplification protocols are employed. In such instances, the initial amplicon composition may be combined with forward and reverse sequencing adaptor primers that include one or more sequencing adaptor domains, e.g., as described above, as well as domains that bind to universal primer sites found in all of the amplicons in the composition, e.g., the forward and reverse anchor domains, such as described above. As reviewed above, amplification conditions may include the addition of forward and reverse sequencing adaptor primers configured to bind to the common forward and reverse anchor domains and thereby amplify all or a desired portion of the product nucleic acid, dNTPs, and a polymerase suitable for effecting the amplification (e.g., a thermostable polymerase for polymerase chain reaction), where examples of such conditions are further described above. The forward and reverse sequencing adaptor primers employed in these embodiments may vary in length, ranging in length in some instances from 20 to 60 nt, such as 25 to 50 nt. Addition of NGS sequencing adaptors results in the production of a composition which is configured for sequencing by an NGS sequencing protocol, i.e., an NGS library.

In certain aspects, the methods of the present disclosure further include subjecting the NGS library to NGS protocol, e.g., as described above. The NGS protocol will vary depending on the particular NGS sequencing system employed. Detailed protocols for sequencing an NGS library, e.g., which may include further amplification (e.g., solid-phase amplification), sequencing the amplicons, and analyzing the sequencing data are available from the manufacturer of the NGS system employed. Protocols for performing next generation sequencing, including methods of processing the sequencing data, e.g., to count and tally sequences and assemble transcriptome data therefrom, are further described in published United States Patent Application 20150344938, the disclosure of which is herein incorporated by reference.

### Pooling

Where desired, a given workflow may include a pooling step where a product composition, e.g., made up of hybridized barcoded gene-specific primer-RNA complexes, synthesized first strand cDNAs or synthesized double stranded cDNAs, is combined or pooled with product compositions obtained from one or more additional samples, e.g., cells. In some instances, for single-cell analysis the pooling step is performed just after hybridization step between barcoded gene-specific primers and target nucleic acids, e.g., as reviewed above. The number of different product compositions produced from different samples, e.g., cells, that are combined or pooled in such embodiments may vary, where the number ranges in some instances from 2 to 1,000,000, such as 3 to 200,000, including 4 to 100,000 such as 5 to 50,000, where in some instances the number ranges from 100 to 10,000, such as 1,000 to 5,000. Prior to or after pooling, the product composition(s) can be amplified, e.g., by polymerase chain reaction (PCR), such as described above.

### Gene-Specific Primer Protocols

As reviewed above, in some instances gene-specific reverse and forward primers may be employed. Aspects of such embodiments include employing a set of gene specific primer pairs, wherein each pair of gene specific primers is made up of a forward primer and a reverse primer, at least one of which includes a sample barcode domain. Examples of sets of reverse gene-specific primers and their use in single cell genetic analysis applications are disclosed in U.S. patent application Ser. Nos. 15/133,184 and 16/543,211, the disclosures of which sets of gene specific reverse primers are incorporated herein by reference.

### Utility

The subject methods find use in a variety of applications, including expression profiling or transcriptome determination applications, where a sample is evaluated to obtain an expression profile of the sample. By “expression profile” is meant the expression level of a gene of interest in a sample, which may be a single cell or a combination of multiple cells (e.g., as determined by quantitating the level of an RNA or protein encoded by the gene of interest), or a set of expression levels of a plurality (e.g., 2 or more) of genes of interest. In certain aspects, the expression profile includes expression level data for 1, 2 or more, 5 or more, 10 or more, 20 or more, 50 or more, 100 or more, 200 or more, 300 or more, 400 or more, 500 or more, 1,000 or more, 5,000 or more, 10,000 or more, 15,000 or more, e.g., 18,000 or more genes of interest. According to one embodiment, the expression profile includes expression level data of from 50 to 8000 genes of interest, e.g., from 1000 to 5000 genes of interest. In some embodiments, the expression profile includes expression level data of from 50 to 19,000 genes of interest, e.g., from 1000 to 18,000 genes of interest. In certain aspects, the methods may be employed detecting and/or quantitating the expression of all or substantially all of the genes transcribed by an organism, e.g., a mammal, such as a human or mouse, in a target cell. The terms “expression” and “gene expression” include transcription and/or translation of nucleic acid material. For example, gene expression profiling may include detecting and/or quantitating one or more of any RNA species transcribed from the genomic DNA of the target cell, including pre-mRNAs, mRNAs, non-coding RNAs, microRNAs, small RNAs, regulatory RNAs, and any combination thereof.

Expression levels of an expressed sequence are optionally normalized by reference or comparison to the expression level(s) of one or more control expressed genes, including but not limited to, ACTB, GAPDH, HPRT-1, RPL25, RPS30, and combinations thereof. These “normalization genes” have expression levels that are relatively constant among target cells in the cellular sample.

In some instances, quantitative analysis of gene expression using set of calibration control template composition is performed. Internal calibration control templates which mimic but differ from natural target RNAs and spiked into cell or cell lysates at specific amount may be effectively used for truly quantitative expression analysis. The calibration control RNAs could be developed for the set of genes (e.g. cell marker genes) or for genome-wide set of transcripts. In order to address the reproducibility of the profiling assay for multiple biological samples (e.g. thousands of single cells), embodiments of the invention uniquely employ the strategy of using barcoded reverse gene specific primers. Target template RNAs (e.g., present in cell extracts) hybridized with barcoded reverse gene specific primers could be combined for the all follow-up steps. The strategy of barcoding and combining target RNAs at early (hybridization) stage allows for significantly reduced cost of the assay, eliminates sample-to-sample profiling variability due to differences in experimental assay conditions, etc. The developed protocol which addresses sample-to-sample and batch effect variability has significant utility in biomarker discovery in clinical samples (e.g., whole blood).

According to certain embodiments, the expression profile includes “binary” or “qualitative” information regarding the expression of each gene of interest in a target cell. That is, in such embodiments, for each gene of interest, the expression profile only includes information that the gene is expressed or not expressed (e.g., above an established threshold level) in the sample being analyzed, e.g., tissue, cell, etc. In other embodiments, the expression profile includes quantitative information regarding the level of expression (e.g., based on rate of transcription, rate of splicing and/or RNA abundance) of one or more genes of interest. A qualitative and/or quantitative expression profile from the sample may be compared to, e.g., a comparable expression profile generated from other samples and/or one or more reference profiles from cells known to have a particular genotype, biological phenotype or condition (e.g., cellular DNA with a specific natural or engineered mutation, a disease condition, such as a tumor cell; or treatment condition, such as a cell treated with an agent, e.g., a drug). When the profiles being compared are quantitative expression profiles, the comparison may include determining a fold-difference between one or more genes in the expression profile of a target cell and the corresponding genes in the expression profile(s) of one or more different target cells in the cellular sample, or the corresponding genes in a reference cell or cellular sample. Alternatively, or additionally, the expression profile may include information regarding the relative expression levels of different genes in a single target cell. In certain aspects, the fold difference in intercellular expression levels or intracellular expression levels can be determined to be 0.1 or more, 0.5 fold or more, 1 fold or more, 1.5 fold or more, 2 fold or more, 2.5 fold or more, 3 fold or more, 4 fold or more, 5 fold or more, 6 fold or more, 7 fold or more, 8 fold or more, 9 fold or more, or more than 10 fold or more, for example.

In some instances, the methods may be employed to determine the transcriptome of a sample. The term “transcriptome” is employed in its conventional sense to refer to the set of all messenger RNA molecules in one cell or a population of cells. In some instances, a transcriptome includes the amount or concentration of each RNA molecule in addition to the molecular identities. The methods described herein may be employed in detecting and/or quantitating the expression of all genes or substantially all genes of the transcriptome of an organism, e.g., a mammalian organism, such as a human or a mouse, for a particular target cell or a population of cells.

Expression profiles obtained using methods of the invention may be employed in a variety of applications. For example, an expression profile may be indicative of the biological condition of the sample or host from which the sample is obtained, including but not limited to a disease condition (e.g., a cancerous condition, metastatic potential, an epithelial mesenchymal transition (EMT) characteristic, and/or any other disease condition of interest), the condition of the cell in response to treatment with any physical action (e.g., heat shock, hypoxia, normoxia, hydrodynamic stress, radiation, and/or the like), the condition of the cell in response to treatment with chemical compounds (e.g., drugs, cytotoxic agents, nutrients, salts, and/or the like) or biological extracts or entities (e.g., viruses, bacteria, other cell types, growth factors, biologics, and/or the like), and/or any other biological condition of interest (e.g. immune response, senescence, inflammation, motility, and/or the like).

Embodiments of the invention find further application in tumor microenvironment analysis applications. Transcriptome data obtained, e.g., as described above, may be employed to determine the cellular composition of a tumor sample, e.g., to provide an evaluation of the types of cells present in a tumor sample, such as infiltrating hematopoietic cells, tumor cells and bulk tissue cells. For example, transcriptome data may be employed to assess whether a tumor sample does not include infiltrating immune cells, including those of the adaptive and/or innate immune system, such as but not limited to: T, B, natural killer, monocyte, granulocytes, neutrophils, basophils, platelets, and their myeloid and lymphoid progenitor cells, hematopoietic stem cells, and the like. Such information may be used, e.g., in therapy determination applications, for example where the presence of infiltrating immune cells indicates that a patient will be responsive to immunotherapy while the absence of infiltrating immune cells indicates that a patient will not be responsive to immunotherapy. As such, aspects of the invention include methods of therapy determination, where a patient tumor sample is evaluated to assess the tumor microenvironment. Aspects of the invention may further include making a determination to employ an immunotherapy protocol is made if the tumor microenvironment includes infiltrating tumor cells and a determination is made to employ a non-immunotherapy treatment regimen if the tumor microenvironment lacks infiltrating immune cells.

Methods as described here also find use in large-scale profiling of single-cell phenotypes derived from model system (e.g., cultivated cells, organoid cultures, 3D cultures, etc.), model organisms (e.g., mice, rat, monkey, etc.) and clinical samples derived from normal or pathological conditions (e.g., blood, biopsy, sputum, saliva, etc.). Currently, there is a substantial need for comprehensive characterization of different cell types present in normal and pathological conditions. The disclosed methods and compositions provide an improved technological platform for large-scale discovery of key cellular markers for developing novel diagnostic and prognostic tools.

Transcriptome data, e.g., produced as described above, also finds use in other non-clinical applications, such as predictive and prognostic biomarker discovery applications, evaluation of cancer immunoediting mechanism applications, drug target discovery, and the like.

In other embodiments, the gene expression level measurement can be combined with profiling of genotype or genetic changes in the target cells. Genetic changes of interest include both natural changes, e.g., those present in cells derived from biological sources, and engineered modifications in target cells, e.g., in genomic DNA. Examples of natural mutation are single nucleotide polymorphism (SNP), copy number variation (CNV), deletions, translocation, gene fusions, recombinations, etc., which may be associated with development of disease state (e.g., cancer, genetic diseases, etc.) in normal cells. Engineered genetic changes may be generated by a wide range of genetic engineered methods (e.g., delivery of constructs by viral, plasmid vectors, synthetic DNA and RNA constructs, etc.) and include, but are not limited to, gene editing (base editing, homologous recombination, etc.), delivery and expression of effector constructs (sgRNA, shRNA, peptides, proteins, aptamers, microRNA, asRNA, etc.) and the like. Usually, effector constructs could change expression (e.g., activation, repression, inactivation, etc.) of target genes. Other types of genetic constructs which do not change expression of genes but may be employed for cell tracking (clonal barcodes or UMI), measure expression of proteins (e.g., antibody-barcoded oligonucleotide constructs), signaling pathway (transcriptional reporter vectors), and other biological processes (e.g., regulation of immune functions, apoptosis, etc.) may also be employed. In some applications the genetic changes may be identified by the disclosed invention in episomal DNA or in genomic DNA, e.g., if a genetic construct is integrated in genomic DNA. In other applications the genetic changes or effector constructs may be transcribed and profiled by designing gene specific primers specific for both effector and transcribed cellular RNAs. Importantly, the disclosed methods of multiplex PCR may generate both expression profile and identify genetic changes or/and effectors in a single assay and therefore characterize and link the phenotype of the cells with specific genetic changes. For example, the combination of expression profiles with identification of effectors (sgRNA, shRNA, etc.) which could induce knock-out, knock-down or activation of target genes allows one to characterize the functions of these genes in normal and disease state. Simultaneous profiling of the natural or induced mutations and transcriptome allows one to find and characterize mechanisms of driver mutations critical for development of disease states (e.g., cancer, senescence, etc.). Monitoring cell phenotypes by expression profiling of different cell clones (e.g., with different mutations labelled by different barcodes) under different growth conditions (cellular environment, drug treatment) allows one to identify rare cancer stem or drug resistant cells.

### Compositions

Aspects of the invention further include various compositions. Compositions of the invention may include, e.g., one or more of any of the reaction mixture components described above with respect to the subject methods. For example, the compositions necessary for generation of cell/barcoded bead complexes may include individual cells or group of cells, barcoded beads with a cell binding moiety, buffers necessary for binding and purification of cell/barcoded bead complexes, and the like. In some embodiments, additional components comprising consumables and reagents (designed for binding physically separated cell/barcoded bead complexes to a solid surface, like plastic, may be included in composition. The composition necessary for generation of primed template nucleic acids may include components like polymers necessary for formation of stimulus responsive polymers (e.g., methylcellulose), cell media (e.g., PBS), hybridization buffer (e.g., 1×TCL, etc.) and lysis buffer (e.g., 0.2% NP-40) as detailed above. Additional components which could be used to increase efficiency, specificity and rate of cell lysis, hybridization (e.g., salts or polynucleotides) and barcoded primer releasing reagents (e.g., DTT) may also be included in the composition. The components necessary for pooling and purification of primed template nucleic acids including oligo dT magnetic beads, AMPure beads, washing and elution buffers, etc. may be included in compositions. Compositions necessary for generation of barcoded nucleic acids and barcoded amplicon compositions may include a primed template nucleic acid, polymerase (e.g., a reverse transcriptase and thermostable DNA polymerase), dsDNAse, single-stranded nuclease (e.g. exonuclease I) a set of gene specific, anchor PCR and indexed NGS primers, dNTPs, a polymerase, buffers, a metal cofactor, one or more nuclease inhibitors (e.g., an RNase inhibitor), one or more enzyme-stabilizing components (e.g., DTT), or any other desired reaction mixture component(s). Composition may vary for the different steps of the disclosed methods. For example, for cDNA synthesis steps the compositions may include only reagents necessary for reverse transcription (e.g., reverse transcriptase) and for the subsequent primer extension and amplification step the composition may employ a different buffer, oligonucleotides and enzymes (DNA polymerase) components. Some components of composition (e.g., barcoded oligonucleotides), may be immobilized on a solid surface (e.g., plate wall, beads, etc.), employed in solution or deposited in microtiter plate. Also provided are compositions that include a barcoded primer extension product composition, e.g., as described above. Also provided are barcoded amplicon compositions and NGS libraries, such as described above.

The subject compositions may be present in any suitable environment. According to one embodiment, the compositions are present in reaction tubes (e.g., a 0.2 mL tube, a 0.5 mL tube, a 1.5 mL tube, or the like), a well (e.g. 6-, 24-, or 96-well plates), and a vials (e.g., 5, 10, 50, 200 mL bottles). In certain aspects, the compositions are present in two or more (e.g., a plurality of) reaction tubes or wells (e.g., a plate, such as a 96-well plate). The tubes and/or plates and/or vials may be made of any suitable material, e.g., polypropylene, or the like. In certain aspects, the tubes and/or plates in which the composition is present provide for efficient heat transfer to the composition (e.g., when placed in a heat block, water bath, thermocycler, and/or the like), so that the temperature of the composition may be altered within a short period of time, e.g., as necessary for a particular cell lysis, hybridization, or enzymatic reaction to occur. According to certain embodiments, the composition is present in a thin-walled polypropylene tube, or a plate having thin-walled polypropylene wells. Other suitable environments for the subject compositions include, e.g., a microfluidic chip (e.g., a “lab-on-a-chip device”). The composition may be present in an instrument(s) configured to analyze composition of cell/barcoded bead complexes (e.g., microscope with image analysis functions), treat the composition with physical stimulus (e.g., UV light) or bring the composition to a desired temperature, e.g., a temperature-controlled water bath, heat block, or the like. The instrument configured to bring the composition to a desired temperature may be configured to bring the composition to a series of different desired temperatures, each for a suitable period of time (e.g., the instrument may be a thermocycler).

### Kits

Aspects of the present disclosure also include kits. The kits may include, e.g., one or more of any of the reaction mixture components described above with respect to the subject methods. For example, the kits may include one or more of: a set of gene specific primers, barcoded oligonucleotides (e.g., barcoded reverse gene specific primers immobilized on the beads), a polymerase (e.g., a thermostable polymerase, a reverse transcriptase both with hot-start properties, or the like), dsDNAse, exonuclease, dNTPs, a metal cofactor, one or more nuclease inhibitors (e.g., an RNase inhibitor and/or a DNase inhibitor), one or more molecular crowding agents (e.g., polyethylene glycol, or the like), one or more enzyme-stabilizing components (e.g., DTT), a stimulus response polymer, or any other desired kit component(s), such as solid supports, containers, cartridges, e.g., tubes, beads, plates, microfluidic chips, etc.

Components of the kits may be present in separate containers, or multiple components may be present in a single container. For example, the individual barcoded oligonucleotides could be provided pre-aliquoted in separate wells or attached/encapsulated with different beads, and mixture of all barcoded beads is provided as kit components. In certain embodiments, it may be convenient to provide the components in a lyophilized form, so that they are ready to use and can be stored conveniently at room temperature.

In addition to the above-mentioned components, a subject kit may further include instructions for using the components of the kit, e.g., to practice the subject method. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, Hard Disk Drive (HDD), portable flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The following examples are offered by way of illustration and not by way of limitation.

### EXPERIMENTAL

### Example 1. Protocol for Producing Sets of Experimentally Validated Gene-Specific Primers

### 1A. Introduction.

Primer design is a complex and unsolved problem. To this end, as described in patent application Ser. Nos. 15/133,184 and 15/914,895 in a more detail (the disclosures of which applications are herein incorporated by reference, we describe the development of a novel in silico multiplex primer design pipeline to unambiguously access primer quality—defined here as the ability to efficiently and specifically amplify the desired template fragments in a complex reaction—on the basis of the primer sequences, target template and the reference/background genome sequence. Subsequently, we used the aforementioned resource and experimentally validated all PCR primers, resulting in multiplex PCR primers with uniform properties.

Briefly, our primer design pipeline consists of four major steps: (1) identify all primer binding-site positions among all possible DNA/RNA target template sequences; (2) evaluate the binding stability of the entire primer sequence using the thermodynamic model to calculate the duplex stability; (3) filter amplicons by size and target region position and (4) in silico designed primer pairs are experimentally validated using primers/corresponding target regions and used under a common PCR thermal profile, facilitating the evaluation of target transcripts of a large number genes in parallel using Next Generation Sequencing (NGS).

General rules concerning optimal primer length, CG content, annealing and melting temperature, and secondary structure issues were included. Since oligonucleotide primers are hypothesized to be specific and provide the optimal annealing and melting temperatures, primers of 18-30 nt were considered to be the best for forward gene specific primer target sequence extension reactions in target regions and GC content of >50% <75%. Importantly, reverse gene specific primers designed for the RNA/DNA hybridization step and follow-up extension step are usually longer (e.g., 30-40 nt) in order to provide higher stability of hybridization complex between target template and primer.

### 1B. Design and Synthesis of Barcoded Reverse and Forward Gene Specific and Oligo dT Primers.

Barcoded reverse gene specific primers were assembled by ligation of a pool of reverse gene specific primers with barcoded oligonucleotides immobilized on the surface of beads:

Link1s
(SEQ ID NOs: 09 and 20)
3′ L2 5′3′ L1 Anchor2 5′
RevGSP-ACCGACCAGCACCpGCCAGCACGCCA-(Barcode)-AGACAC
GACCAGCCACGAGCA-X-Bead A-TGGCTGGTCGTGG--CGGTCGTGCG
GT-3′dT

Barcoded oligonucleotides with the minimum structure: linker 5′-Anchor 2-Barcode-Linker L1-3′ are ligated to a reverse gene specific primer set (RevGSP) with a minimum structure 5′-phosphate-Linker L2-RevGSP-3′ using complementary to linker L1 and linker L2 oligonucleotide Link1s and DNA ligase under ligation conditions. DNA ligation reaction attaches the barcoded anchor oligonucleotides to the reverse gene specific primers. In an alternative strategy, antisense RevGSP-L1 complement oligonucleotides are synthesized, annealed to L1-barcoded oligonucleotides and extended by Klenow DNA polymerase (or thermostable DNA polymerase). As a result of the ligation (or primer extension) reaction, the set of reverse gene specific primers are labelled with specific barcodes.

In alternative design, the reverse gene specific primer domain is replaced for a dT30VN domain, wherein V is G or C or A, and N is G, or C, or A or T. Both linker ligation by T4 DNA ligase and primer extension by DNA polymerase protocols may be employed to produce anchored and barcoded oligo dT primers.

The set of barcoded reverse gene specific (or oligo dT) primers is purified from non-ligated products by washing the bead-barcoded RevGSP (oligo dT) conjugates in 0.1M of NaOH and used in disclosed primer extension assay. The same set of gene specific primers may be labelled with plurality of different barcodes using the same protocol. In another embodiment, the same protocol may be used for barcoding set of forward gene specific primers.

Barcode-Anchor oligonucleotide may be attached to the solid surface (e.g. beads) through linker X (e.g., X could be a cleavable linker). Furthermore, the different binding moiety (e.g., antibodies) may be attached to the beads to provide binding of Antibody-Bead-barcoded GSP complex to specific cell types through antigen-antibody interaction.

Importantly, each barcode may have a complex structure as described above in more detail. These complex composite barcodes could have several domains, including but not limited to:
1) Sample barcode—specific sequence (usually from 8-14 nt) attached to a set of gene-specific primers which allow to label all extension products derived from target RNA sample.
2) Universal molecular identifier (UMI)—complex random, semi-random (usually 8-12 nt), or set of unique specific sequences which allow to label each molecule used in disclosed primer extension assay with unique sequence/barcode. UMI could be added to RevGSP-Linker 2 set between RevGSP and linker L2.
3) Bead barcode—specific sequence (10-16 nt) unique for each bead if gene-specific primers are attached to the beads. In some embodiments, e.g., for single cell analysis applications (e.g. if only one biological sample used in the assay) bead barcode could be sample barcode.
4) Antibody barcode—specific sequence unique for the each specific antibody immobilized to the beads.
   Linker L1, linker L2 and complementary Link1s could be designed with variety of different sequences with minimum length of L1 and L2 are 4 nt each.

### Examples of Anchor2-Barcode-Linker 1 Oligonucleotides Used in Ligation Reaction:

Barcodes are underlined

| | Anc2-BC1-L1 |
|---|---|
| (SEQ ID NO: 11) | |
| | ACGAGCACCGACCAGCACAGAGAACAAACACCGCACGACCG |
| | Anc2-BC2-L1 |
| (SEQ ID NO: 12) | |
| | ACGAGCACCGACCAGCACAGAGGCGAAACACCGCACGACCG |
| | Anc2-BC3-L1 |
| (SEQ ID NO: 13) | |
| | ACGAGCACCGACCAGCACAGAGCAAAAGGACCGCACGACCG |

### Example of Bead-Barcoded Oligonucleotide Conjugates (Synthesized by Chemgenes, Inc.) Used in Ligation Reaction.

In the diagram below: PClinker—photocleavable linker, or SSlinker—bi-sulfite linker cleaved by sulfite ions (e.g., DTT treatment) is used for detachment of reverse barcoded gene specific primers from the beads; Anchor2—binding site for universal amplification primer; UMI—universal molecular index; Barcode—sample-specific 6 nt barcode (underlined); Linker L2—sequence necessary for ligation of barcodes with gene specific primer set; bead—polystyrene or hydrogel beads with sizes 10-100 microns.

(SEQ ID NO: 14 and 15)
ChemB-PC1-Anc2-BC-L2
Anchor 2 UMI Barcode Linker L2
Bead-linker-PClinker-AGCACCGACCAGCACAGAVVNVVNVVCAT
CAGACCGCACGACCG-3′
ChemB-SS-Anc2-BC-L2
Anchor 2 UMI Barcode Linker L2
Bead-linker-SSlinker-AGCACCGACCAGCACAGAVVNVVNVVCAG
CATGACCGCACGACCG-3′

### Example of Final Barcoded Reverse Gene Specific Primer Structure Employed in the Assay

Anchor2 L1-L2 linker
(SEQ ID NO: 16)
5′-ACGAGCACCGACCAGCACAGA-(UMI-Barcode)-ACCGCACGACC
GCCACGACCAGCCA-RevGSP-3′
Anchor2 L1-L2 linker
(SEQ ID NO: 17)
5′-ACGAGCACCGACCAGCACAGA-(UMI-Barcode)-ACCGCACGACC
GCCACGACCAGCCA-dT30VN-3′

Wherein, L2-L1 linker sequence generated by ligation of L1 and L2 linkers, Barcode—complex barcode, and UMI—universal molecular index as described in more details above, Anchor2—universal primer binding site.

In some embodiments, the barcoded reverse gene specific primer composition could be synthesized by a combinatorial (pool and split) chemical synthesis protocol without DNA ligation step. In this embodiment, L2-L1 linker will be missing in the final structure.

A similar structure could be generated for barcoded forward gene specific primer set and employed in the disclosed assay:

Anchor1 L1-L2 linker
(SEQ ID NO: 18)
5′-AGCACCGACCAGCAGACA-(UMI-Barcode)-ACCGCACGACCGCC
ACGACCAGCCA-RevGSP-3′

In other embodiment, the forward gene specific primers are designed and used in the assay without barcodes and synthesized by conventional oligonucleotide synthesis with the following structure:

| | Anchor1 |
|---|---|
| (SEQ ID NO: 19) | |
| | 5′-AGCACCGACCAGCAGACA-FwdGSP-3′ |

### 1C. High-throughput Gene Specific Primer Validation

Multiplex PCR primers with cognate target sequences were screened en masse. In some embodiments, the set of barcoded reverse gene specific primers (with the structure shown above) is first hybridized to control natural or synthetic template RNAs. Furthermore, the hybrids between target mRNA and barcoded reverse gene specific hybrids are combined together, purified and used as a mix in the follow-up primer extension and amplification steps. In some embodiments, the hybridization step is performed with RNA sample and barcoded reverse gene specific primers in solution (e.g., primers released from beads). As discussed in a more detail above, the selection of primers with high hybridization efficiency and stability of target mRNA-primer complexes is a desired step which defines the overall performance of the assay and cross-talk between different samples. Moreover, using the barcoded reverse primers in the first step of the protocol allows one to combine all samples together and therefore scale-up the assay for analysis of hundreds-thousands of samples in the single test tube format.

In another embodiment, the natural or synthetic template RNAs are reverse transcribed e.g., from barcoded oligo dT primers, and synthesized cDNAs are used as templates for the extension step using forward gene specific primers and follow-up amplification steps.

In both protocols. uniformity of amplification, including primer efficiency, primer specificity and dynamic range (minimum 100-fold) is determined from multiplex reaction kinetic data. In order to reliably measure expression of different genes, a panel of 10 different human universal RNA from different commercial sources (e.g., Agilent, Clontech, BioChain, Qiagen, etc.) and synthetic template RNA is used as templates for cDNA synthesis. Non-specific primer activities are measured by yield of non-targeted products from human universal RNAs and negative control templates (human genomic DNA and mouse universal RNAs). The protocol for testing primer performance is repeated several times with set of 3-5 PCR primer pairs per gene until the primers with high specific and low non-specific activity were selected. Finally, functionally validated primers are selected as experimentally validated primers for use in sets of experimental validated gene specific primers.

### Example 2. Development of Barcoded Beads with Cell Binding Moiety

To develop barcoded beads with cell binding moiety several strategies are employed. In the first approach, to chimeric oligonucleotide with structure:

| | L2 |
|---|---|
| (SEQ ID NO: 20) | |
| | 5′-pCCACGACCAGCCA-Moiety |

is synthesized by conventional phosphoramidite chemistry and ligated to barcoded beads using the protocol described in a more detail above. Examples of cell binding moieties which are synthesized as oligonucleotide conjugates include lipids (cholesterol, fatty acid, like stearoylic or palmitic acid, oligonucleotide aptamers, e.g., CD4 aptamer with structure:

(SEQ ID NO: 21)

5′-CCACCACCGTACAATTCGCTTTCTTTTTTCATTACCTACTCTGGC-3′

In the second approach, the non-specific (e.g., against beta2-microglobulin, CD293) or cell-type specific (e.g., against CD4, CD8, CD19, etc.) antibodies are conjugated to barcoded beads through covalent or non-covalent bonds. In one protocol, the antibodies with cell binding properties are bound to beads (e.g., polystyrene beads) though passive adsorption. In other protocols, antibodies are bound to beads using amino-modified linker domain (with structure: 5′-pCCACGACCAGCCA-NH2-3′) (SEQ ID NO: 22) ligated to barcoded beads) and click chemistry. In this protocol the antibodies and amino-modified barcoded beads are activated and conjugated using click chemistry (ThunderLink kit, Expedion). In another protocol, the amino-modified oligonucleotide complementary to L21-L2 linker domain:

| (SEQ ID NO: 23) | |
|---|---|
| | 5′NH2-TGGCTGGTCGTGGCGGTCGTGCGGT-3′ |

is conjugated with antibodies using conventional click chemistry regents and protocol (ThunderLink kit, Expedion). The antibody-Linker complement conjugates are incubated with barcoded beads in buffer comprising 50 mM TrisHCl, ph 7.8, 1 M NaCl, 0.1% Tween20 at 12° C. for 3 hours and purified from non-bind antibodies by using washing in 1×PBS solution and centrifugation steps.

### Example 3. Generation of Barcoded Bead-Cell Complexes by Binding of Barcoded Beads with Cell Sample and Enrichment for Single Cell-Single Bead Complexes in Solution

The barcoded beads with cell binding moiety are washed in 1×PBS and bind with single cells at ratio 1.5-2/1 in 1×PBS solution in rotating test tubes at 37° C. for 30 minutes. The single barcoded bead-cell complexes are purified from larger cell-bead complexes by filtration through cell strainer (cell sieve with 40 or 100 micron pores) or by FACS (Becton Dickenson FACS Melody) based on forward and side scattering characteristics. FACS purification allows one to separate single bead-cell complexes from targeting both multiple bead-cell complexes, empty bind beads and unbound cells. Moreover, FACS allows one to purify only one or several specific cell type-barcoded bead complexes if barcoded beads have cell-type specific binding moiety (e.g., antibodies for CD8 for T cells, or CD19 for B cells, etc.).

### Example 4. Binding of Barcoded Beads and Cells on Solid Support

A single cell suspension of HEK293 cells (1×10⁶ cells, control and activated by TNF) transduced with barcoded lentiviral sgRNA library (80 sgRNAs targeting genes involved in NFkB signaling pathway) is bound to cell culture plastic dish (20-cm diameter) and incubated overnight in cell culture media (DMEM). In another protocol, the plastic surface is modified by spotting of micro patterning areas (e.g., 10-20 microns) separated from each other (e.g., 100-200 microns) of cell adhesion ligands (e.g., collagen, fibronectin, etc.) in a way that facilitates attachment of single cells in a spaced apart manner. The cells randomly attached to plastic are washed in 1×PBS and bound with Antibody-barcoded bead conjugates (beta2-microglobulin and CD293 antibody bead conjugates) comprising a set of 180 reverse gene-specific primers specific for genes involved and regulated by NFkB signaling pathway. The barcoded antibody-bead conjugates are incubated with plastic-attached beads in plate shaker in 1×PBS for 30 minutes and cell-barcoded bead complexes attached to plastic surface are purified from non-attached beads by washing in 1×PBS buffer.

### Example 5. Generation of Barcoded Primed Nucleic Acid Template by Hybridization of Barcoded Reverse Gene Specific Primers with Cellular Target RNAs

The example protocol below describes methods for expression profiling of PBMC cells in 3D methylcellulose matrix or cells immobilized on a solid support. As a starting material, the protocol may employ any single cell suspension of interest in a 1×PBS buffer at 1-10×10⁶ per ml or cells attached to plastic surface (see Example 4 protocol) at density of approximately 200-1000 cells per square cm. In addition the protocol may use beads (e.g., 20-40 micron polystyrene beads) with covalently attached via a photocleavable linker barcoded reverse gene specific primers designed for 1.7K cell marker genes (or barcoded oligo dT primer specific for polyA+RNAs), and non-covalently attached antibodies specific to cell surface (anti-beta-2-microglobulin, anti-CD298) as described in Examples 1 and 2. Furthermore, the protocol is based on the use of stimulus-responsive free matrix: a matrix substance whose physical state can be altered by a stimulus to immobilize bead-cell complexes in a 3-D matrix or on the plastic surface so as to allow spatially limited cell lysis, release of RNA or DNA and hybridization of cellular RNA/DNA to barcoded gene specific primers provided by bead linked to a given single cell through the cell binding moiety. Examples of matrices include methylcellulose prepared as 5-10% gels in PBS which solidify (‘gel’) upon heating to temperatures in the 45-60° C. range. Furthermore, the cell lysis/hybridization solution is used to lyse the cells in cell/bead complexes scattered in the matrix and promote hybridization of cellular nucleic acids with barcoded oligonucleotides. As an example, Qiagen TCL buffer can be used at 0.5-2× concentration with additional components, like 1% sarcosine, 1% CTAB, 1% NP40, NaCl (e.g. 0.5M), 10% PEG, proteinase K.

Protocol Steps for Isolation of Leukocytes from Normal Human PBMC Sample:
1. Isolate PBMC from anticoagulated human blood sample using Ficoll-based density gradient centrifugation (e.g., SepMate tubes, StemCell Technologies)
2. Wash PBMC and resuspend in PBS at concentration of 1-10×10⁶ per ml.
3. Incubate PBMC with barcoded beads comprising attached cell binding antibodies (against beta2-microglobulin and CD293) at room temperature for 10-30 minutes
4. Cell suspension containing bead-cell complexes is analyzed by flow cytometry to identify single cell-bead complexes by forward and side light scatter bivariate plot; this subset is sorted into 1×PBS aiming for a concentration of 30-90K cells per ml.
5. Sorted barcoded bead-cell complexes are mixed with stimulus-responsive matrix, e.g., for 2 replicates of 1 ml matrix each, prepare 3 ml of methylcellulose (prepared in 1×PBS) so as to achieve final concentration of methylcellulose of 6-9% containing 1-10K cells per 1 ml of gel. This step is done at room temperature where the methylcellulose solution is a viscous liquid capable of mixing with barcoded bead-cell complex containing solutions.
6. Pour 1 ml of matrix with bead-cell complexes into 1 well of 6 well plate, repeat for desired number of replicates; smaller or larger volumes can be prepared for use with similarly smaller or large volume wells in 24 well or single large plates.
7. Swirl plates to assure uniform distribution of matrix within well.
8. Place plate in hot water bath set to 45-60° C. on platform to allow only lower 5 mm of plate to contact hot water. Allow plates to gel for 5-20 minutes. Gelification is visible as a milky white change in methylcellulose matrices.
9. After initial gelification is complete, remove plate lid to allow addition of 0.5-2 ml of cell lysis/hybridization buffer (preheated to same temperature as used in bath) onto surface of gelified matrix. Swirl to assure uniform and complete coverage of well.
10. Expose open plate to UV 365 light for 5-15 minutes to cleave photo-sensitive linker, thereby releasing barcoded reverse gene specific primers from beads
11. Continue incubation after removing UV light for 10-40 minutes to allow hybridization of cellular RNA (or if necessary DNA) released by lysis with barcoded reverse gene specific provided by the bead of the bead-cell complex
12. After hybridization incubation, remove plate to room temperature. Tilt plate and aspirate liquid lysis buffer on surface of gel using pipette and suction.
13. Add 50 microliters of magnetic oligo-dT beads (Thermo-Fisher) in in 1 ml of 1×TCL buffer (Qiagen) to each well. In another protocol, the primed barcoded RNA hybrid was bind to and purified using AMPure magnetic beads (0.8× volume) using manufacturer's protocol (Beckman-Coulter).
14. Shake plate at 300-600 rpm for 20-30 minutes.
15. Collect liquid from each well, centrifuge to pellet oligo dT-barcoded Reverse gene specific primer-RNA complexes (primed barcoded RNA).
16. Wash the oligo dT with bind primed barcoded RNA three times with 1×TCW washing buffer (Qiagen) using magnetic stand.
17. Proceed to multiplex RT-PCR and NGS.

### Example 6. Multiplex RT-PCR Assay

### 6A. Design of Primers for Anchor Addition, First and Second PCR Steps

Design of Barcoded Forward and Barcoded Reverse gene specific primers with anchor1 (Fwd-anchor1-GSP primers) and anchor2 (Rev-anchor2-GSP primers) with 3′-extended suppression portions for primer extension steps and universal PCR primers (F-MP1GAC and R-MP2CAG) to amplify anchored cDNA fragments by PCR.

Sequences that are underlined are the common PCR suppression portions, and those in italics and bold are unique sequences for Fwd or Rev primers, respectively, and GSP is the gene-specific primer domain. The BC-Link is Barcode-Linker domain which comprise the composite barcode as describes in more details above and could be present in only reverse (preferred embodiment), only in forward or in both reverse and forward primers.

| (SEQ ID NOs: 24 to 25) | |
|---|---|
| | F-MP1GAC |
| | AGCAGCACCGACCAGCAGAC |
| | AGCACCGACCAGCAGACA(BC-Link)FwdGSP> |
| | Fwd-Anc1-GSP cDNA Rev-Anc2-GSP |
| | <RevGSP(Link-BC)AGACACGACCAGCCACGA |
| | GACACGACCAGCCACGAGCA |
| | R-MP2CAG |

For simplicity the structures below show the design of primers and amplification products only for the preferred embodiment of using barcoded reverse and non-barcoded forward gene specific primer set:

| (SEQ ID NOs: 26 to 27) | |
|---|---|
| | F-MP1GAC |
| | AGCAGCACCGACCAGCAGAC |
| | AGCACCGACCAGCAGACA-FwdGSP> |
| | Fwd-Anc1-GSP cDNA Rev-Anc2-GSP |
| | <RevGSP(BC-Link)AGACACGACCAGCCACGA |
| | GACACGACCAGCCACGAGCA |
| | R-MP2CAG |

The resultant structure of amplified cDNA products after the two sequential primer extension steps using Barcoded Rev-anchor2-GSPs using RNA as a template and Fwd-anchor1-GSPs using barcoded cDNA template and a first PCR step using universal F-MP1GAC and R-MP2CAG primers is shown below:

(60-250nt)
(SEQ ID NOs: 28 to 29)
AGCAGCACCGACCAGCAGACA-FwdGSP-cDNA-RevGSP-Link-BC-T
CTGTGCTGGTCGGTGCTCGT
TCGTCGTGGCTGGTCGTCTGT-FwdGSP-cDNA-RevGSP-Link-BC-A
GACACGACCAGCCACGAGCA

The amplified products after first PCR cDNA are then subjected to a second round of PCR to add IIlumina P7, P5 sequencing adaptors. PCR primers for the second PCR step comprise anchor 1 and anchor 2 binding domains, indexing (highlighted in red) domains (optional domains, can be used if experiment requires to combine the different samples together for NGS step) and P5 or P7 sequences necessary for cluster formation in Illumina NGS instrument, as illustrated below:

Set of Forward Indexing Primers for 2^{nd} PCR
step:
(SEQ ID NOS: 30 to 35)
FP7-A1Ind-A
AGCAGAAGACGGCATACGAGATATACGACAGCAGCAGCACCGACCAGCAG
ACA
FP7-A1Ind-B
AGCAGAAGACGGCATACGAGATACTGATGAGCAGCAGCACCGACCAGCAG
ACA
FP7-A1Ind-C
AGCAGAAGACGGCATACGAGATAGCATCAAGCAGCAGCACCGACCAGCAG
ACA
FP7-A1Ind-D
AGCAGAAGACGGCATACGAGATAAGTGGTAGCAGCAGCACCGACCAGCAG
ACA
FP7-A1Ind-E
AGCAGAAGACGGCATACGAGATATCGGATAGCAGCAGCACCGACCAGCAG
ACA
FP7-A1Ind-F
AGCAGAAGACGGCATACGAGATACATAGCAGCAGCAGCACCGACCAGCAG
ACA
Set of Reverse Indexing Primers for 2^{nd} PCR
step:
(SEQ ID NOS: 36 to 41)
RP5-A2Ind-A
ACGGCGACCACCGAGATCTACACATACGACACGACGAGCACCGACCAGCA
CAGA
RP5-A2Ind-B
ACGGCGACCACCGAGATCTACACACTGATGACGACGAGCACCGACCAGCA
CAGA
RP5-A2Ind-C
ACGGCGACCACCGAGATCTACACAGCATCAACGACGAGCACCGACCAGCA
CAGA
RP5-A2Ind-D
ACGGCGACCACCGAGATCTACACAAGTCGTACGACGAGCACCGACCAGCA
CAGA
RP5-A2Ind-E
ACGGCGACCACCGAGATCTACACATCGCATACGACGAGCACCGACCAGCA
CAGA
RP5-A2Ind-F
ACGGCGACCACCGAGATCTACACACATAGCACGACGAGCACCGACCAGCA
CAGA
Set of Forward and Reverse Non-indexing
Primers for 2^{nd} PCR step:
(SEQ ID NOS: 42 to 43)
FP7-A1
AGCAGAAGACGGCATACGAGATAGCAGCAGCACCGACCAGCAGACA
RP5-A2
ACGGCGACCACCGAGATCTACACACGACGAGCACCGACCAGCACAGA

After a second PCR step with Forward and Reverse indexing primers the final amplicon structure, flanked with P7 and P5 IIlumina's adaptor sequences, and ready for NGS step is shown below:

(SEQ ID NOs: 44 to 45)

P7(Ind)AGCAGCACCGACCAGCAGACA-FwdGSP-cDNA-RevGSP
(LinkBC)TCTGTGCTGGTCGGTGCTCGT(Ind)P5
P7(Ind)TCGTCGTGGCTGGTCGTCTGT-FwdGSP-cDNA-RevGSP
(LinkBC)AGACACGACCAGCCACGAGCA(Ind)P5

The sequences of primers for NGS sequencing (e.g. IIlumina NextSeq500 platform) of cDNA inserts, barcode domain and indexes are provided below:

| | SeqDNAlink-Rev |
|---|---|
| (SEQ ID NO: 46) | |
| | TGGCGTGCTGGCGGTGCTGGTCGGT |
| | SeqDNA-Fwd |
| (SEQ ID NO: 47) | |
| | AGCAGCAGCACCGACCAGCAGACA |
| | SeqBarcode-Fwd |
| (SEQ ID NO: 48) | |
| | ACCGACCAGCACCGCCAGCACGCCA |
| | Optional sequencing primers: |
| | SeqIND-Fwd |
| (SEQ ID NO: 49) | |
| | TCTGTGCTGGTCGGTGCTCGTCGT |
| | SeqIND-Rev |
| (SEQ ID NO: 50) | |
| | TGTCTGCTGGTCGGTGCTGCTGCT |
| | SeqDNA-Rev |
| (SEQ ID NO: 51) | |
| | ACGACGAGCACCGACCAGCACAGA |

An example of protocol for NGS sequencing of amplified cDNA products in Next Seq500 machine using 150-nt sequencing kit is shown below:

Read 1: SeqDNAlink-Rev>81 cycles
Ind 1: SeqIND-Rev>6 cycles
Ind 2: SeqBarcode-Fwd>38 cycles
Read 2: SeqDNA-Fwd>35 cycles
The read number for SeqBarcode-Fwd primer could depend of the design of specific barcode domain cassette. The number of read 38 was selected for reading complex sample barcode domain with the structure: Antibody barcode(6)-Sample barcode(6)-Bead barcode(14)-UMI(12).
6B. Protocol for Multiplex RT-PCR Amplification of Target Genes for Expression Profiling or Mutation Analysis or Combined Expression-Effector Analysis Starting from Barcoded Primed RNA Template (See Example 5).

Step 1. Barcoded primed RNAs (purified as pooled hybrid between RNA and barcoded reverse gene specific primer from thousands of cell-barcoded bead complexes as in Example 5) is treated with Exonuclease I (10 units) in 104 of reaction mix containing 1×GC buffer, dNTP (500 uM) at 37° C. for 15 min and converted to barcoded cDNA by adding Maxima Reverse Transcriptase (200 units, Thermo-Fisher) and incubating the reaction mix at 50° C. for 30 min and 95° C. for 5 min.

Step 2. Barcoded cDNA is primed (add universal anchors 1) using mix of Forward-anchor1-GSP primers (5 nM final concentration for each primer) in 20-ul reaction mix comprising 1×GC buffer, dNTP (250 μM) and Phusion II (4 units, Thermo-Fisher) for 1 cycles at (98° C. for 1 min, 64° C. for 30 min) and treated with exonuclease I (1 μl, 10 units, New England Biolabs) at 37° C. for 30 min.

Step 3. 1^{st} PCR step. Whole volume (20-μl) of barcoded anchored cDNA fragments (from Step 2) are amplified in 75-μl reaction mix comprising 1×GC Buffer, dNTP (200 μM), universal PCR primers F-MP1GAC and R-MP2CAG and Phusion II (15 units, Thermo-Fisher) for 18-20 cycles (starting from 2,000 cell-barcoded bead complexes) at (98° C. for 10 sec, 72° C. for 20 sec).

Step 4. 2^{nd} PCR step. 5-μl aliquot of 1st PCR is amplified in 100-μl of PCR mix comprising 1×GC Buffer, dNTP (200 μM), indexed (specific for the each of several samples) or non-indexed (only for one sample) Fwd and Rev PCR primers and Phusion II (20 units, Thermo-Fisher) for 7 cycles at (98° C. for 10 sec, 72° C. for 20 sec).

Step 5. The amplified PCR products are analyzed in 3.5% agarose-1×TAE gel to optimize the cycle number and finally digested with exonuclease I (20 units, New England Biolabs), incubated and 37° C. for 30 min, inactivated at 65° C. for 15 min and purified in Qia PCR column. Purified PCR products were quantitated by Qubit (Thermo-Fisher) and if necessary different samples were mixed together (at equal amount), diluted to 10 nM and sequenced in NextSeq500 using Illumina paired-end protocol and reagents for 150 cycles.

### 6C. Protocol for Multiplex RT-PCR Amplification of Target Genes for Expression Profiling, in Single Cells Using Barcoded Oligo dT Primer.

A wide range of conventional protocols (see reference section below) may be employed by using barcoded oligo dT primers for gene specific (using set of forward gene specific primers, see example 5) or unbiased genome-wide (for all polyA+RNAs) compartment free expression profiling at the single cell level.

Step 1. Barcoded primed RNAs (purified as pooled hybrid between RNA and barcoded oligo dT primer from thousands of cell-barcoded bead complexes as in Example 5) is treated with Exonuclease I (10 units) in 10-μl of reaction mix containing 1×GC buffer, dNTP (500 uM) at 37° C. for 15 min and converted to barcoded cDNA by adding Maxima Reverse Transcriptase (200 units, Thermo-Fisher) and incubating the reaction mix at 50° C. for 30 min and 95° C. for 5 min.

Step 2A. For targeted gene specific expression profiling, the barcoded cDNA is primed (add universal anchors 1) using a mix of Forward-anchor1-GSP primers designed in close proximity from polyA tail for any specific set of genes (5 nM final concentration for each primer) in 204 reaction mix comprising 1×GC buffer, dNTP (250 uM) and Phusion II (4 units, Thermo-Fisher) for 1 cycles at (98° C. for 1 min, 64° C. for 30 min) and treated with exonuclease I (1 μl, 10 units, New England Biolabs) at 37° C. for 30 min. All follow-up step for 1^{st}, 2^{nd} amplification and NGS sequencing as in Example 5.

Step 2B Alternative protocol for genome-wide expression profiling of polyA+RNA is based on conventional RNAseq protocols. Example protocol include but not limited to Nextera XT protocol (based on adding sequencing adaptors using Tn5 transposase) (https://supportillumina.com/sequencing/sequencing_kits/nextera_xt_dna_kit/documentation.html) or TrueSeq Stranded RNA protocol: https://support.illumina.com/downloads/truseq_stranded_total_rna_sample_preparation_guide_15031048.htm Moreover, a wide range of other protocols, described in reference section, could be employed for generation of adaptor ligated cDNA products for NGS step.

### 7. REFERENCE SECTION

(See U.S. Provisional Application Ser. No. 62/895,719 filed on Sep. 4, 2019, the disclosure of which is herein incorporated by reference.

Notwithstanding the appended claims, the disclosure is also defined by the following clauses:

1. A compartment-free method of preparing barcoded nucleic acids, the method comprising:

combining a cellular sample with a plurality of distinct barcoded beads comprising barcoded reverse primers under conditions sufficient to produce a liquid composition comprising a plurality of separated cell/barcoded bead complexes;

hybridizing template binding domains of barcoded reverse primers to template nucleic acids of the cells to produce primed template nucleic acids; and

subjecting the primed template nucleic acids to primer extension reaction conditions sufficient to produce barcoded nucleic acids.

2. The method according to Clause 1, wherein the cell/barcoded bead complexes are separated from each other by distance of 100 microns or longer.
3. The method according to Clause 2, wherein the cell/barcoded bead complexes are separated from each other by distance of 500 microns or longer.
4. The method according to Clause 3, wherein the cell/barcoded bead complexes are separated from each other by distance of 1000 microns or longer.
5. The method according to any of the preceding clauses, wherein the cell/barcoded bead complexes comprise complexes made up of a single cell or component thereof and a single bead.
6. The method according to any of Clauses 1 to 4, wherein the cell/barcoded bead complexes comprise complexes made up of two cells or components thereof and a single bead.
7. The method according to Clauses 5 or 6, wherein the cell/barcoded bead compositions comprise complexes made up of a cell nucleus or cell nuclei and barcoded beads.
8. The method according to any of the preceding clauses, wherein the cellular sample comprises cells genetically modified by genetic construct.
9. The method according to Clause 8, wherein the genetic construct encodes a clonal barcode.
10. The method according to Clause 9, wherein genetic construct is effector molecule.
11. The method according to Clause 10 wherein the effector molecule is selected from group consisting of: sgRNA, shRNA, microRNA, aptamer, ribozyme, native and mutated peptide, and proteins.
12. The method according to Clause 11, wherein genetic construct is transcribed in the cellular sample.
13. The method according to Clause 12, wherein gene-specific primers designed for the genetic construct are employed.
14. The method according to any of the preceding clauses, wherein the cellular sample is combined with the plurality of distinct barcoded beads in the presence of stimulus responsive polymer that solidifies in response to an applied stimulus.
15. The method according to any of Clauses 1 to 13, wherein the cellular sample is combined with the plurality of distinct barcoded beads in a manner such that the resultant cell/barcoded bead complexes are separated from each other on a surface of a solid support.
16. The method according to Clause 15, wherein the method further comprises applying a medium comprising a stimulus responsive polymer to the surface of the solid support prior to hybridizing template binding domains of barcoded reverse primers to template nucleic acids of the cells to produce primed template nucleic acids.
17. The method according to any of Clauses 14 to 16, wherein the applied stimulus comprises a physical stimulus.
18. The method according to Clause 17, wherein the physical stimulus is a temperature change.
19. The method according to Clause 18, the temperature change comprises a change of 30° C. or greater.
20. The method according to any of Clauses 14 to 16, wherein the applied stimulus comprises a chemical stimulus.
21. The method according to any of Clauses 14 to 20, wherein stimulus responsive polymer is reversible.
22. The method according to any of Clauses 14 to 21, wherein the stimulus responsive polymer comprises a methylcellulose stimulus responsive polymer.
23. The method according to any of Clauses 14 to 22, wherein the method comprises applying the stimulus prior to hybridizing template binding domains of barcoded reverse primers to template nucleic acids of the cells to produce primed template nucleic acids.
24. The method according to any of the preceding clauses, wherein the method comprises releasing the barcoded reverse primers from the barcoded beads prior to hybridizing template binding domains of barcoded reverse primers to template nucleic acids of the cells to produce primed template nucleic acids.
25. The method according to Clause 24, wherein the barcoded reverse primers are bound to the beads by a cleavable linker.
26. The method according to any of the preceding clauses, wherein the method comprises lysing cells of cell-barcoded bead complexes prior to hybridizing template binding domains of barcoded reverse primers to template nucleic acids of the cells to produce primed template nucleic acids.
27. The method according to any of the preceding clauses, wherein the barcoded beads comprise a cellular binding moiety.
28. The method according to Clause 27, wherein the cellular binding moiety comprises an aptamer.
29. The method according to Clause 27, wherein the cellular binding moiety comprises a lipid.
30. The method according to Clause 27, wherein the cellular binding moiety comprises a proteinaceous specific binding member.
31. The method according to Clause 30, wherein the proteinaceous specific binding member comprises an antibody or binding fragment thereof.
32. The method according to any of the preceding clauses, wherein the template binding domains are oligo dT domains.
33. The method according to any of Clauses 1 to 31, wherein the template binding domains are gene specific domains.
34. The method according to Clause 33, wherein the barcoded beads comprise 100 or more distinct gene specific barcoded reverse primers.
35. The method according to any of the preceding clauses, wherein the barcoded reverse primers further comprise an anchor domain.
36. The method according to any of the preceding clauses, wherein the barcoded reverse primers further comprise a unique molecular identifier (UMI) domain.
37. The method according to any of the preceding clauses, wherein the method further comprises amplifying the barcoded nucleic acids to produce an amplified nucleic acid composition.
38. The method according to Clause 37, wherein the amplifying comprises primer extension from a plurality of forward gene specific primers that comprise an anchor domain and a template binding domain complementary to the barcoded nucleic acids.
39. The method according to any of the preceding clauses, wherein the method further comprises sequencing the amplified nucleic acid composition.
40. The method according to Clause 39, wherein the sequence is performed by a NGS protocol.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc.

As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A compartment-free method of preparing barcoded nucleic acids, the method comprising:
combining a cellular sample with a plurality of distinct barcoded beads comprising barcoded reverse primers under conditions sufficient to produce a liquid composition comprising a plurality of separated cell/barcoded bead complexes;
hybridizing template binding domains of barcoded reverse primers to template nucleic acids of the cells to produce primed template nucleic acids; and
subjecting the primed template nucleic acids to primer extension reaction conditions sufficient to produce barcoded nucleic acids.

2. The method according to claim 1, wherein the cell/barcoded bead complexes comprise complexes made up of:
a single cell or component thereof and a single bead; or
two cells or components thereof and a single bead; or
a cell nucleus or cell nuclei and barcoded beads.

3. The method according to any of the preceding claims, wherein the cellular sample comprises cells genetically modified by genetic construct.

4. The method according to claim 3, wherein the genetic construct encodes a clonal barcode.

5. The method according to any of the preceding claims, wherein the cellular sample is combined with the plurality of distinct barcoded beads in the presence of stimulus responsive polymer that solidifies in response to an applied stimulus.

6. The method according to any of claims 1 to 4, wherein the cellular sample is combined with the plurality of distinct barcoded beads in a manner such that the resultant cell/barcoded bead complexes are separated from each other on a surface of a solid support.

7. The method according to any of the preceding claims, wherein the method comprises releasing the barcoded reverse primers from the barcoded beads prior to hybridizing template binding domains of barcoded reverse primers to template nucleic acids of the cells to produce primed template nucleic acids.

8. The method according to any of the preceding claims, wherein the method comprises lysing cells of cell-barcoded bead complexes prior to hybridizing template binding domains of barcoded reverse primers to template nucleic acids of the cells to produce primed template nucleic acids.

9. The method according to any of the preceding claims, wherein the barcoded beads comprise a cellular binding moiety.

10. The method according to any of the preceding claims, wherein the template binding domains are oligo dT domains or gene specific domains.

11. The method according to any of the preceding claims, wherein the barcoded reverse primers further comprise an anchor domain.

12. The method according to any of the preceding claims, wherein the barcoded reverse primers further comprise a unique molecular identifier (UMI) domain.

13. The method according to any of the preceding claims, wherein the method further comprises amplifying the barcoded nucleic acids to produce an amplified nucleic acid composition.

14. The method according to any of the preceding claims, wherein the method further comprises sequencing the amplified nucleic acid composition.

15. The method according to claim 14, wherein the sequence is performed by a NGS protocol.


SEQUENCE LISTING (54 sequences)

SEQ ID NO: 1 - DNA, 18 bp, Artificial sequence
  synthetic sequence
agcaccgaccagcagaca

SEQ ID NO: 2 - DNA, 18 bp, Artificial sequence
  synthetic sequence
agcaccgaccagcacaga

SEQ ID NO: 3 - DNA, 20 bp, Artificial sequence
  synthetic sequence
aatgatacggcgaccaccga

SEQ ID NO: 4 - DNA, 24 bp, Artificial sequence
  synthetic sequence
caagcagaagacggcatacgagat

SEQ ID NO: 5 - DNA, 33 bp, Artificial sequence
  synthetic sequence
acactctttccctacacgacgctcttccgatct

SEQ ID NO: 6 - DNA, 34 bp, Artificial sequence
  synthetic sequence
gtgactggagttcagacgtgtgctcttccgatct

SEQ ID NO: 7 - DNA, 30 bp, Artificial sequence
  synthetic sequence
ccatctcatccctgcgtgtctccgactcag

SEQ ID NO: 8 - DNA, 23 bp, Artificial sequence
  synthetic sequence
cctctctatgggcagtcggtgat

SEQ ID NO: 9 - DNA, 13 bp, Artificial sequence
  synthetic sequence
  misc_feature (1)..(1): The nucleotide is attached to a phosphate
  misc_feature (13)..(13): The nucleotide is attached to RevGSP
ccacgaccagcca

SEQ ID NO: 10 - DNA, 33 bp, Artificial sequence
  synthetic sequence
  misc_feature (1)..(1): The nucleotide is attached to X-Bead
  misc_feature (21)..(22): A barcode is present between the nucleotides
acgagcaccgaccagcacagaaccgcacgaccg

SEQ ID NO: 11 - DNA, 41 bp, Artificial sequence
  synthetic sequence
acgagcaccgaccagcacagagaacaaacaccgcacgaccg

SEQ ID NO: 12 - DNA, 41 bp, Artificial sequence
  synthetic sequence
acgagcaccgaccagcacagaggcgaaacaccgcacgaccg

SEQ ID NO: 13 - DNA, 41 bp, Artificial sequence
  synthetic sequence
acgagcaccgaccagcacagagcaaaaggaccgcacgaccg

SEQ ID NO: 14 - DNA, 44 bp, Artificial sequence
  synthetic sequence
  misc_feature (1)..(1): The nucleotide is attached to a PClinker which is attached to a linker which is attached to a bead
  misc_feature (21)..(21): n is a, c, g, or t
  misc_feature (24)..(24): n is a, c, g, or t
agcaccgaccagcacagavvnvvnvvcatcagaccgcacgaccg

SEQ ID NO: 15 - DNA, 45 bp, Artificial sequence
  synthetic sequence
  misc_feature (1)..(1): The nucleotide is attached to an SSlinker which is attached to a linker which is attached to a bead
  misc_feature (21)..(21): n is a, c, g, or t
  misc_feature (24)..(24): n is a, c, g, or t
agcaccgaccagcacagavvnvvnvvcagcatgaccgcacgaccg

SEQ ID NO: 16 - DNA, 46 bp, Artificial sequence
  synthetic sequence
  misc_feature (21)..(22): UMI-barcode is present between the nucleotides
  misc_feature (46)..(46): The nucleotide is attached to RevGSP
acgagcaccgaccagcacagaaccgcacgaccgccacgaccagcca

SEQ ID NO: 17 - DNA, 46 bp, Artificial sequence; identical to SEQ ID NO: 16
  synthetic sequence
  misc_feature (21)..(22): UMI-Barcode is present between the nucleotides
  misc_feature (46)..(46): The nucleotide is attached to dT30VN

SEQ ID NO: 18 - DNA, 43 bp, Artificial sequence
  synthetic sequence
  misc_feature (18)..(19): UMI-Barcode is present between the nucleotides
  misc_feature (43)..(43): The nucleotide is attached to RevGSP
agcaccgaccagcagacaaccgcacgaccgccacgaccagcca

SEQ ID NO: 19 - DNA, 18 bp, Artificial sequence
  synthetic sequence
  misc_feature (18)..(18): The nucleotide is attached to FwdGSP
agcaccgaccagcagaca

SEQ ID NO: 20 - DNA, 13 bp, Artificial sequence
  synthetic sequence
  misc_feature (13)..(13): The nucleotide is attached to a moiety
ccacgaccagcca

SEQ ID NO: 21 - DNA, 45 bp, Artificial sequence
  synthetic sequence
ccaccaccgtacaattcgctttcttttttcattacctactctggc

SEQ ID NO: 22 - DNA, 13 bp, Artificial sequence
  synthetic sequence
  misc_feature (1)..(1): The nucleotide is attached to a phosphate
  misc_feature (13)..(13): The nucleotide is attached to NH2
ccacgaccagcca

SEQ ID NO: 23 - DNA, 25 bp, Artificial sequence
  synthetic sequence
  misc_feature (1)..(1): The nucleotide is attached to NH2
tggctggtcgtggcggtcgtgcggt

SEQ ID NO: 24 - DNA, 20 bp, Artificial sequence
  synthetic sequence
agcagcaccgaccagcagac

SEQ ID NO: 25 - DNA, 18 bp, Artificial sequence
  synthetic sequence
  misc_feature (18)..(18): The nucleotide may be attached to (BC-Link) FwdGSP
agcaccgaccagcagaca

SEQ ID NO: 26 - DNA, 18 bp, Artificial sequence
  synthetic sequence
  misc_feature (1)..(1): The nucleotide is attached to (Link-BC) which is attached to RevGSP
agacacgaccagccacga

SEQ ID NO: 27 - DNA, 20 bp, Artificial sequence
  synthetic sequence
gacacgaccagccacgagca

SEQ ID NO: 28 - DNA, 42 bp, Artificial sequence
  synthetic sequence
  misc_feature (21)..(22): FwdGSP-cDNA-RevGSP-Link-BC is present between the nucleotides
agcagcaccgaccagcagacatctgtgctggtcggtgctcgt

SEQ ID NO: 29 - DNA, 42 bp, Artificial sequence
  synthetic sequence
  misc_feature (21)..(22): FwdGSP-cDNA-RevGSP-Link-BC is present between the nucleotides
tcgtcgtggctggtcgtctgtagacacgaccagccacgagca

SEQ ID NO: 30 - DNA, 53 bp, Artificial sequence
  synthetic sequence
agcagaagacggcatacgagatatacgacagcagcagcaccgaccagcagaca

SEQ ID NO: 31 - DNA, 53 bp, Artificial sequence
  synthetic sequence
agcagaagacggcatacgagatactgatgagcagcagcaccgaccagcagaca

SEQ ID NO: 32 - DNA, 53 bp, Artificial sequence
  synthetic sequence
agcagaagacggcatacgagatagcatcaagcagcagcaccgaccagcagaca

SEQ ID NO: 33 - DNA, 53 bp, Artificial sequence
  synthetic sequence
agcagaagacggcatacgagataagtcgtagcagcagcaccgaccagcagaca

SEQ ID NO: 34 - DNA, 53 bp, Artificial sequence
  synthetic sequence
agcagaagacggcatacgagatatcgcatagcagcagcaccgaccagcagaca

SEQ ID NO: 35 - DNA, 53 bp, Artificial sequence
  synthetic sequence
agcagaagacggcatacgagatacatagcagcagcagcaccgaccagcagaca

SEQ ID NO: 36 - DNA, 54 bp, Artificial sequence
  synthetic sequence
acggcgaccaccgagatctacacatacgacacgacgagcaccgaccagcacaga

SEQ ID NO: 37 - DNA, 54 bp, Artificial sequence
  synthetic sequence
acggcgaccaccgagatctacacactgatgacgacgagcaccgaccagcacaga

SEQ ID NO: 38 - DNA, 54 bp, Artificial sequence
  synthetic sequence
acggcgaccaccgagatctacacagcatcaacgacgagcaccgaccagcacaga

SEQ ID NO: 39 - DNA, 54 bp, Artificial sequence
  synthetic sequence
acggcgaccaccgagatctacacaagtcgtacgacgagcaccgaccagcacaga

SEQ ID NO: 40 - DNA, 54 bp, Artificial sequence
  synthetic sequence
acggcgaccaccgagatctacacatcgcatacgacgagcaccgaccagcacaga

SEQ ID NO: 41 - DNA, 54 bp, Artificial sequence
  synthetic sequence
acggcgaccaccgagatctacacacatagcacgacgagcaccgaccagcacaga

SEQ ID NO: 42 - DNA, 46 bp, Artificial sequence
  synthetic sequence
agcagaagacggcatacgagatagcagcagcaccgaccagcagaca

SEQ ID NO: 43 - DNA, 47 bp, Artificial sequence
  synthetic sequence
acggcgaccaccgagatctacacacgacgagcaccgaccagcacaga

SEQ ID NO: 44 - DNA, 42 bp, Artificial sequence; identical to SEQ ID NO: 28
  synthetic sequence
  misc_feature (1)..(1): The nucleotide is attached to (Ind) which is attached to p7
  misc_feature (21)..(22): FwdGSP-cDNA-RevGSP(LinkBC) is present between the nucleotides
  misc_feature (42)..(42): The nucleotide is attached to (Ind)p5

SEQ ID NO: 45 - DNA, 42 bp, Artificial sequence; identical to SEQ ID NO: 29
  synthetic sequence
  misc_feature (1)..(1): The nucleotide is attached to (Ind) which is attached to p7
  misc_feature (21)..(22): FwdGSP-cDNA-RevGSP(LinkBC) is present between the nucleotides
  misc_feature (42)..(42): The nucleotide is attached to (Ind)p5

SEQ ID NO: 46 - DNA, 25 bp, Artificial sequence
  synthetic sequence
tggcgtgctggcggtgctggtcggt

SEQ ID NO: 47 - DNA, 24 bp, Artificial sequence
  synthetic sequence
agcagcagcaccgaccagcagaca

SEQ ID NO: 48 - DNA, 25 bp, Artificial sequence
  synthetic sequence
accgaccagcaccgccagcacgcca

SEQ ID NO: 49 - DNA, 24 bp, Artificial sequence
  synthetic sequence
tctgtgctggtcggtgctcgtcgt

SEQ ID NO: 50 - DNA, 24 bp, Artificial sequence
  synthetic sequence
tgtctgctggtcggtgctgctgct

SEQ ID NO: 51 - DNA, 24 bp, Artificial sequence
  synthetic sequence
acgacgagcaccgaccagcacaga

SEQ ID NO: 52 - DNA, 26 bp, Artificial sequence
  synthetic sequence
  misc_feature (26)..(26): The nucleotide is attached to deoxythymine
atggctggtcgtggcggtcgtgcggt

SEQ ID NO: 53 - DNA, 18 bp, Artificial sequence
  synthetic sequence
  misc_feature (18)..(18): The nucleotide is attached to FwdGSP
agcaccgaccagcagaca

SEQ ID NO: 54 - DNA, 18 bp, Artificial sequence
  synthetic sequence
  misc_feature (1)..(1): The nucleotide is attached to (BC-Link) which is attached to RevGSP
agacacgaccagccacga